(19) United States
(12) Patent Application Publication
(10) Pub. No.: US 2004/0147522 A1
(43) Pub. Date: Jul. 29, 2004

(54) Compounds having both alpha7 nicotinic agonist activity and 5HT3 antagonist activity for the treatment of CNS diseases

(76) Inventors: Erik Ho Fong Wong, Portage, MI (US); Luz A. Cortes-Burgos, Saint Charles, MO (US); Bruce Nelson Rogers, Mystic, CT (US); David W. Piotrowski, Groton Long Point, CT (US); Daniel Patrick Walker, Noank, CT (US); Eric Jon Jacobsen, Chesterfield, MO (US); Brad A. Acker, Chesterfield, MO (US); Donn G. Wishka, Groton Long Point, CT (US)

Correspondence Address: PHARMACIA & UPJOHN, 301 HENRIETTA ST, 0228-32-LAW, KALAMAZOO, MI, 49007, US

(21) Appl. No.: 10/698,227

(22) Filed: Oct. 31, 2003

Related U.S. Application Data
(60) Provisional application No. 60/423,155, filed on Nov. 1, 2002.

(51) Int. Cl. A61K 31/498; A61K 31/4745; A61K 31/46

(52) U.S. Cl. 514/249; 514/304; 514/305; 514/303; 544/349; 546/113; 546/125; 546/135

(57) ABSTRACT

The invention discloses compounds that are selective α7 nAChR agonists and 5-HT₃ antagonists. The compounds are useful for treating many CNS diseases.

### CROSS REFERENCE TO RELATED APPLICATIONS

This application claims the benefit of U.S. provisional application Serial No. 60/423155 filed on 1 Nov. 2002, under 35 USC 119(e)(i), which is incorporated herein by reference in its entirety.

### FIELD OF INVENTION

The present invention relates to molecules that have a greater effect upon the α7 nAChRs as compared to other closely related members of this large ligand-gated receptor family and are simultaneously 5-HT₃ antagonists. Thus, the invention provides compounds that are active drug molecules with fewer side effects.

### BACKGROUND OF THE INVENTION

5-Hydroxytryptamine (5-HT) is a very pharmacologically versatile neurotransmitter. It induces activation and/or inhibition of smooth and cadiac muscle, exocrine and endocrine glands, central and peripheral neurons and cells of the mematopoietic and immune systems (for review see Fozard & Saxena, 1991; Serotonin: Molecular Biology, Receptors and Functional Effects, Basel, Birkhauser). The basis of this versatility is the existence of multiple receptor sites of which seven are generally recognized based on genetic, second message coupling and pharmacological critieria (Hoyer et al., 1994; Pharmacol Rev, 46, 157-203). The 5-HT₃ receptor is unique among mono- and di-amine neurotransmitter receptors in not being coupled via a G protein to its effector system. Rather, it is a ligand gated ion channel (Derkach et al 1989; Nature, 339, 706-709), and is formed of multiple subunits of molecular weight lower than typically expected for a G-protein coupled receptor. In this context, it is analogous to the nicotinic, GABA_{A} and glycine receptors.

The development of potent, selective and specific 5-HT₃ receptor antagonists allow the demonstration of behavorial effects in rodents and primates suggestive of central actions (Costall et al, 1990; Pharmacol Ther, 47, 181-202). Autoradiographic studies in human brain tissue indicated 5-HT₃ binding sites in forebrain structures and in the medulla oblongata are localized in essentially the same structures as that observed in rat studies. Effects of these antagonists in a variety of animal models of CNS disorders suggest utility for the treatment of chemotherapy-induced emesis, anxiety, schizophrenia, psychosis, dementia, drug dependence, diarrhoea associate with carcinoid syndrome and pain.

Nicotinic acetylcholine receptors (nAChRs) also play a large role in central nervous system (CNS) activity. Particularly, they are known to be involved in cognition, learning, mood, emotion, and neuroprotection. There are several types of nicotinic acetylcholine receptors, and each one appears to have a different role in regulating CNS function. Nicotine affects all such receptors, and has a variety of activities. Unfortunately, not all of the activities are desirable. In fact, one of the least desirable properties of nicotine is its addictive nature and the low ratio between efficacy and safety. The present invention relates to molecules that are selective α7 nAChRs agonists and are simultaneously 5-HT₃ antagonists. Thus, the invention provides compounds that are active drug molecules with fewer side effects.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Recently, Eisele et al. has indicated that a chimeric receptor formed between the N-terminal ligand binding domain of the α7 nAChR (Eisele et al., Nature, 366(6454), p 479-83, 1993), and the pore forming C-terminal domain of the 5-HT₃ receptor expressed well in Xenopus oocytes while retaining nicotinic agonist sensitivity. Eisele et al. used the N-terminus of the avian (chick) form of the α7 nAChR receptor and the C-terminus of the mouse form of the 5-HT₃ gene. However, under physiological conditions the α7 nAChR is a calcium channel while the 5-HT₃R is a sodium and potassium channel. Indeed, Eisele et al. teaches that the chicken α7 nAChR/mouse 5-HT₃R behaves quite differently than the native α7 nAChR with the pore element not conducting calcium but actually being blocked by calcium ions. WO 00/73431 A2 reports on assay conditions under which the 5-HT₃R can be made to conduct calcium. This assay may be used to screen for agonist activity at this receptor.

WO 00/73431 A2 discloses two binding assays to directly measure the affinity and selectivity of compounds at the α7 nAChR and the 5-HT₃R. The combined use of these functional and binding assays may be used to identify compounds that are selective agonists of the α7 nAChR.

Recently, Macor reported (Macor at al. Bioorg & Med Chem Let 11(2001) 319-321) that tropesitron had both α7 nicotinic agonist activity and 5-HT₃ antagonist activity and that the other compounds tested did not posess both activities. Surprisingly, we have found the compounds of the present invention to be both α7 agonists and 5-HT₃ antagonists. Compounds possessing this dual activity offer unique opportunities over compounds that are either α7 agonists or 5-HT₃ antagonists, but not both, to treat one or more or combination of the following diseases or conditions: schizophrenia, psychosis, cognitive and attention deficit symptoms of Alzheimer's, neurodegeneration associated with diseases such as Alzheimer's disease, pre-senile dementia (also known as mild cognitive impairment), senile dementia, traumatic brain injury, behavioral and cognitive problems associated with brain tumors, Parkinson's disease, amyotrophic lateral sclerosis, AIDS dementia complex, dementia associated with Down's syndrome, dementia associated with Lewy Bodies, Huntington's disease, attention deficit disorders, attention deficit hyperactivity disorder, depression, anxiety, general anxiety disorder, post traumatic stress disorder, mood and affective disorders including disruptive and oppositional conditions, borderline personality disorder, panic disorder, tardive dyskinesia, restless leg syndrome, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy, symptoms associated with pain, chemotherapy-induced emesis, migraine, fibromyalgia, irritable bowel syndrome, and diarrhea associated with carcinoid syndrome.

### SUMMARY OF THE INVENTION

The present invention discloses compounds of Formula I that have both α7 nicotinic agonist activity and 5HT₃ antagonist activity. The compound of Formula I is:

Azabicyclo-N(H)—C(═O)—W⁰ Formula I

wherein Azabicyclo is

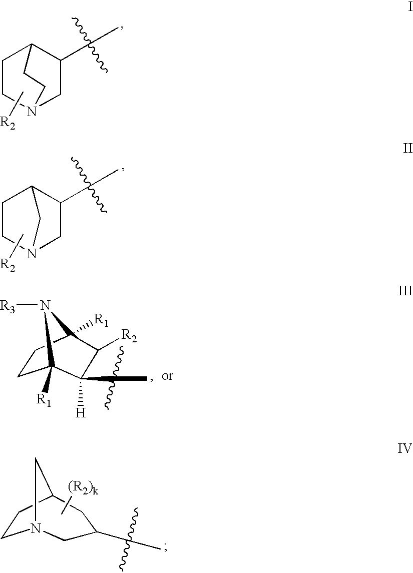

Each R₁ is independently H, alkyl, or substituted alkyl;

Each R₂ is independently H, alkyl, or substituted alkyl;

k is 1 or 2, provided that one R₂ is other than H when k is 2;

R₃ is H, alkyl, or an amino protecting group;

W⁰ is

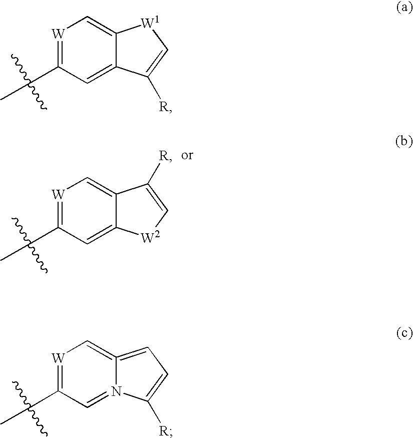

W is CH or N;

W¹ is O, N(R₄), N(C(O)R₄), or S;

W² is O, N(R₄), N(C(O)R₄), or S;

R is H, F, Cl, Br, I, alkyl, substituted alkyl, or alkynyl;

Each R₄ is independently H or alkyl optionally substituted where valency allows with up to 3 substituents independently selected from —OH, —CN, NH₂, —NO₂, —CF₃, F, Cl, Br, or I;

and pharmaceutically acceptable salts thereof.

Embodiments of the invention may include one or more or combination of the following.

One embodiment of the present invention provides a use of a compound of Formula I for treating, or preparing a medicament to treat, a disease or condition, where the diseases, disorders, and/or condition is any one or more or combination of the following: schizophrenia, psychosis, cognitive and attention deficit symptoms of Alzheimer's, neurodegeneration associated with diseases such as Alzheimer's disease, pre-senile dementia (also known as mild cognitive impairment), senile dementia, traumatic brain injury, behavioral and cognitive problems associated with brain tumors, Parkinson's disease, amyotrophic lateral sclerosis, AIDS dementia complex, dementia associated with Down's syndrome, dementia associated with Lewy Bodies, Huntington's disease, attention deficit disorders, attention deficit hyperactivity disorder also known as hyperkinetic disorder, depression, anxiety, general anxiety disorder, post traumatic stress disorder, mood and affective disorders including disruptive and oppositional conditions, borderline personality disorder, panic disorder, tardive dyskinesia, restless leg disorder, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy (e.g., glaucoma and diabetic rentinopathy), symptoms associated with pain (central and peripheral), chemotherapy-induced emesis, migraine, fibromyalgia, irritable bowel syndrome, and diarrhea associated with carcinoid syndrome.

In another aspect, the invention includes treating a mammal suffering from schizophrenia or psychosis by administering compounds of Formula I in conjunction with antipsychotic drugs (also called anti-psychotic agents). The compounds of the present invention and the antipsychotic drugs can be administered simultaneously or at separate intervals. When administered simultaneously the compounds of the present invention and the antipsychotic drugs can be incorporated into a single pharmaceutical composition. Alternatively, two separate compositions, i.e., one containing compounds of the present invention and the other containing antipsychotic drugs, can be administered simultaneously.

The present invention also includes the compounds of the present invention, pharmaceutical compositions containing the active compounds as the free base or as a pharmaceutically acceptable salt and a pharmaceutically acceptable carrier, and methods to treat the identified diseases.

A further embodiment of the present invention provides a method comprising administering a therapeutically effective amount of a compound of the present invention or a pharmaceutical composition contains said compound to the mammal.

Another group of compounds of Formula I includes compounds where R₂ is H. Another group of compounds of Formula I includes compounds where R₂ is H, or alkyl. Another group of compounds of Formula I includes compounds where R₂ is alkyl. Another group of compounds of Formula I includes compounds where R₂ is methyl. Another group of compounds of Formula I includes compounds where R₂ is substituted alkyl. Another group of compounds of Formula I includes compounds where R₂ is benzyl (methyl substituted with phenyl).

Another group of compounds of Formula I includes compounds where Azabicyclo is I, II, III, or IV. Another group of compounds of Formula I includes compounds where W is (a), (b), or (c).

Another group of compounds of Formula I includes compounds where each R₁ is H. Another group of compounds of Formula I includes compounds where one R₁ is H and the other R₁ includes any one of alkyl, or substituted alkyl. Another group of compounds of Formula I includes compounds where each R₁ is independently any one of alkyl, or substituted alkyl.

Another group of compounds of Formula I includes compounds where R₃ is H. Another group of compounds of Formula I includes compounds where R₃ is alkyl. Another group of compounds of Formula I includes compounds where R₃ is an amino protecting group.

Another group of compounds of Formula I includes compounds where W¹ and W² are independently any one or more of the following: O, N(R₄), N(C(O)R₄), or S. Another group of compounds of Formula I includes compounds where R₄ is H. Another group of compounds of Formula I includes compounds where R₄ is alkyl optionally substituted where valency allows with up to 3 substituents independently selected from —OH, —CN, NH₂, —NO₂, —CF₃, F, Cl, Br, or I.

Another group of compounds of Formula I includes compounds where R is any one or more of the following: H, F, Cl, Br, I, alkyl, substituted, or alkynyl. It is preferred that R is F, Cl, Br, I, alkyl including lower alkyl, substituted alkyl including lower substituted alkyl, or alkynyl including lower alkynyl, for example but not by way of limitation, R is F, Cl, Br, I, or alkyl including lower alkyl; R is Br; R is alkyl including lower alkyl; or R is i-propyl.

Another group of compounds of Formula I includes compounds where W is CH and W¹, W², R, R₁, R₂, R₃, and R₄ are as described herein. Another group of compounds of Formula I includes compounds where W is N and W¹, W², R, R₁, R₂, R₃, and R₄ are as described herein. One of ordinary skill in the art will recognize that where alkyl, substituted alkyl or alkynyl is allowed, so is lower alkyl, lower substituted alkyl or lower alkynyl, respectively.

Another group of compounds of Formula I includes compounds where the compound is any one or more or combination of the following as the free base or as a pharmaceutically acceptable salt thereof:

N-[(3R)-1-azabicyclo[2.2.2]oct-3-yl]thieno[3,2-c]pyridine-6-carboxamide

N-[(3S)-1-azabicyclo[2.2.2]oct-3-yl]thieno[3,2-c]pyridine-6-carboxamide

N-[(3R)-1-azabicyclo[2.2.2]oct-3-yl]-3-chlorofuro[2,3-c]pyridine-5-carboxamide

N-[(3S)-1-azabicyclo[2.2.2]oct-3-yl]-3-chlorofuro[2,3-c]pyridine-5-carboxamide

N-[(3R)-1-azabicyclo[2.2.2]oct-3-yl]-3-bromofuro[2,3-c]pyridine-5-carboxamide

N-[(3S)-1-azabicyclo[2.2.2]oct-3-yl]-3-bromofuro[2,3-c]pyridine-5-carboxamide

N-[(3R)-1-azabicyclo[2.2.2]oct-3-yl]-3-bromothieno[2,3-c]pyridine-5-carboxamide

N-[(3R)-1-azabicyclo[2.2.2]oct-3-yl]-6-bromopyrrolo[1,2-a]pyrazine-3-carboxamide

N-[(3R)-1-azabicyclo[2.2.2]oct-3-yl]-6-ethynylpyrrolo[1,2-a]pyrazine-3-carboxamide

N-[(3R)-1-azabicyclo[2.2.2]oct-3-yl]-3-bromo-1-benzofuran-5-carboxamide

N-[(3S)-1-azabicyclo[2.2.2]oct-3-yl]-3-bromo-1-benzofuran-5-carboxamide

N-[(3R)-1-azabicyclo[2.2.2]oct-3-yl]-3-isopropyl-1-benzofuran-5-carboxamide

N-[(3S)-1-azabicyclo[2.2.2]oct-3-yl]-3-isopropyl-1-benzofuran-5-carboxamide

N-[(1S, 2R, 4R)-7-azabicyclo[2.2.1]hept-2-yl]-3-isopropyl-1-benzofuran-5-carboxamide.

In another aspect, the invention includes methods of treating a mammal suffering from schizophrenia or psychosis by administering compounds of Formula I, or preparing a medicament comprising compounds of Formula I, in conjunction with antipsychotic drugs. The compounds of Formula I and the antipsychotic drugs can be administered simultaneously or at separate intervals. When administered simultaneously the compounds of Formula I and the antipsychotic drugs can be incorporated into a single pharmaceutical composition. Alternatively, two separate compositions, i.e., one containing compounds of Formula I and the other containing antipsychotic drugs, can be administered simultaneously.

The compounds of Formula I (Azabicyclo I) have asymmetric centers on the quinuclidine ring. The compounds of the present invention include quinuclidines having 3R configuration, 2S, 3R configuration, or 3S configuration and also include racemic mixtures and compositions of varying degrees of streochemical purities. For example, and not by limitation, compounds of Formula I include compounds with stereospecificity including:

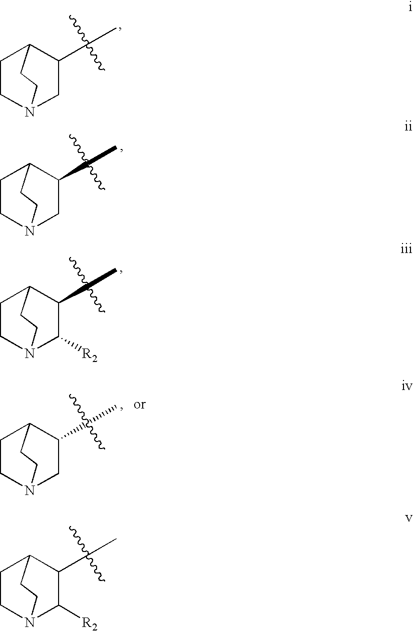

wherein the Azabicyclo

(i) is a racemic mixture;

(ii) has the stereochemistry of 3R at C3;

(iii) has the 3R,2S stereochemistry at C3 and C2, respectively;

(iv) has the stereochemistry of 3S at C3; or

(v) is a racemic mixture; and for (iii) and (v), R₂ has any definition or specific value discussed herein.

The compounds of Formula I (Azabicyclo III) have asymmetric centers on the 7-azabicyclo[2.2.1]heptane ring which can exhibit a number of stereochemical configurations.

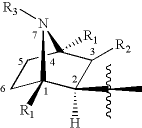

The terms exo and endo are stereochemical prefixes that describe the relative configuration of a substituent on a bridge (not a bridgehead) of a bicyclic system. If a substituent is oriented toward the larger of the other bridges, it is endo. If a substituent is oriented toward the smaller bridge it is exo. Depending on the substitution on the carbon atoms, the endo and exo orientations can give rise to different stereoisomers. For instance, when carbons 1 and 4 are substituted with hydrogen and carbon 2 is bonded to a nitrogen-containing species, the endo orientation gives rise to the possibility of a pair of enantiomers: either the 1S, 2S, 4R isomer or its enantiomer, the 1R, 2R, 4S isomer. Likewise, the exo orientation gives rise to the possibility of another pair of stereoisomers which are diastereomeric and C-2 epimeric with respect to the endo isomers: either the 1R, 2S, 4S isomer or its enantiomer, the 1S, 2R, 4R isomer. The compounds of this invention exist in the exo orientation. For example, when R₂=R₁=H, the absolute stereochemistry is exo-(1S, 2R, 4R).

The compounds of the present invention (Azabicyclo III) have the exo orientation at the C-2 carbon and S configuration at the C-1 carbon and the R configuration at the C-2 and the C-4 carbons of the 7-azabicyclo[2.2.1]heptane ring. Unexpectedly, the inventive compounds exhibit much higher activity relative to compounds lacking the exo 2R, stereochemistry. For example, the ratio of activities for compounds having the exo 2R configuration to other stereochemical configurations may be greater than about 100:1. Although it is desirable that the stereochemical purity be as high as possible, absolute purity is not required. For example, pharmaceutical compositions can include one or more compounds, each having an exo 2R configuration, or mixtures of compounds having exo 2R and other configurations. In mixtures of compounds, those species possessing stereochemical configurations other than exo 2R act as diluents and tend to lower the activity of the pharmaceutical composition. Typically, pharmaceutical compositions including mixtures of compounds possess a larger percentage of species having the exo 2R configuration relative to other configurations.

The compounds of Formula I have asymmetric center(s) on the [2.2.1] azabicyclic ring at C3 and C4. The scope of this invention includes the separate stereoisomers of Formula I being endo-4S, endo-4R, exo-4S, exo-4R:

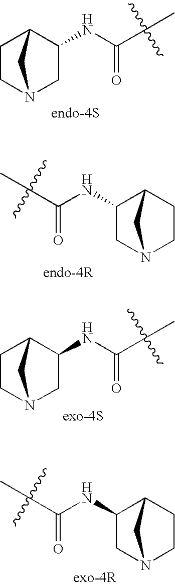

The endo isomer is the isomer where the non-hydrogen substituent at C3 of the [2.2.1] azabicyclic compound is projected toward the larger of the two remaining bridges. The exo isomer is the isomer where the non-hydrogen substituent at C3 of the [2.2.1] azabicyclic compound is projected toward the smaller of the two remaining bridges. Thus, there can be four separate isomers: exo-4(R), exo-4(S), endo-4(R), and endo-4(S). Some embodiments of compounds of Formula I for when Azabicyclo is II include racemic mixtures where R₂ is H or is at C2 or C6; or Azabicyclo II has the exo-4(S) stereochemistry and R₂ has any definition discussed herein and is bonded at any carbon discussed herein, e.g., C2 or C6.

The compounds of Formula I have asymmetric center(s) on the [3.2.1] azabicyclic ring at C3 and C5. The scope of this invention includes the separate stereoisomers of Formula I being endo-3S, 5R, endo-3R, 5S, exo-3R, 5R, exo-3S, 5S:

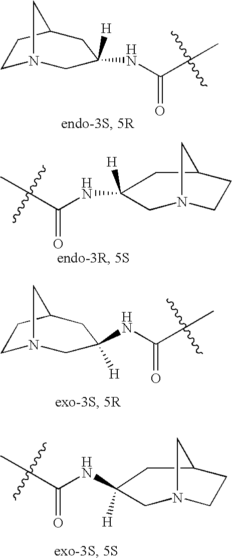

Another group of compounds of Formula I includes any one or more or combination of the following:

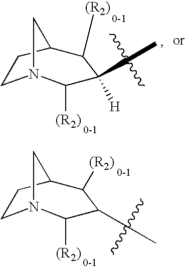

wherein the Azabicyclo has the stereochemistry of 3R, 5R , or is a racemic mixture and where each R₂ can be H or present and have any definition or specific value discussed herein.

Stereoselective syntheses and/or subjecting the reaction product to appropriate purification steps produce substantially optically pure materials. Suitable stereoselective synthetic procedures for producing optically pure materials are well known in the art, as are procedures for purifying racemic mixtures into optically pure fractions.

The compounds of the present invention having the specified stereochemistry above have different levels of activity and that for a given set of values for the variable substitutuents one isomer may be preferred over the other isomers. Although it is desirable that the stereochemical purity be as high as possible, absolute purity is not required. It is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF THE INVENTION

Surprisingly, we have found that compounds of Formula I have both α7 nicotinic agonist activity and 5HT₃ antagonist activity. The compounds of Formula I are:

Azabicyclo-N(H)—C(═O)—W⁰ Formula I

wherein Azabicyclo is

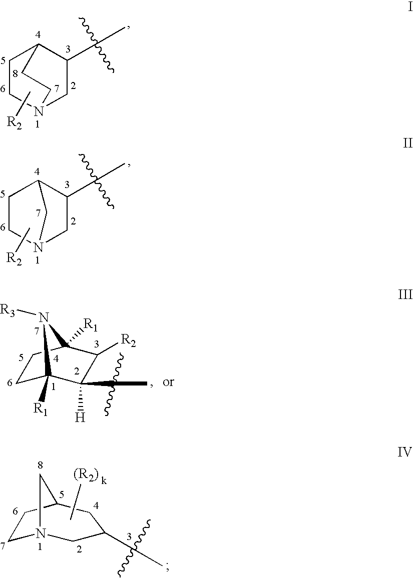

Each R₁ is independently H, alkyl, or substituted alkyl;

Each R₂ is indepently H, alkyl, or substituted alkyl;

k is 1 or 2, provided that one R₂ is other than H when k is 2;

R₃ is H, alkyl, or an amino protecting group;

W⁰ is

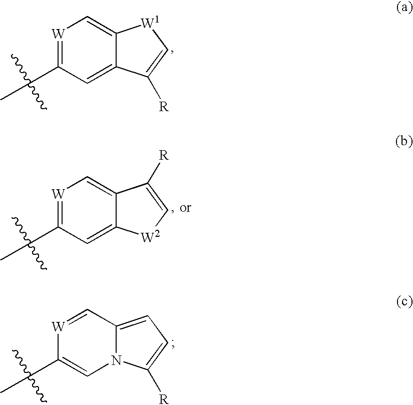

W is CH or N;

W¹ is O, N(R₄), N(C(O)R₄), or S;

W² is O, N(R₄), N(C(O)R₄), or S;

R is H, F, Cl, Br, I, alkyl, substituted alkyl, or alkynyl;

Alkyl is both straight- and branched-chain moieties having from 1-6 carbon atoms;

Substituted alkyl is alkyl having 1-3 substituents independently selected from F, Cl, Br, or I and further optionally having 1 substituent selected from —CN, —NO₂, —CF₃, —OR₄, —SR₄, —S(O)₂R₄, —S(O)R₄, —OS(O)₂R₄, —N(R₄)₂, —C(O)R₄, —C(S)R₄, —C(O)OR₄, —C(O)N(R₄)₂, —N(R₄)C(O)R₄, —N(R₄)C(O)N(R₄)₂, —S(O)₂N(R₄)₂, —N(R₄)S(O)₂R₄, or phenyl, wherein phenyl is optionally substituted with up to 4 substituents independently selected from F, Cl, Br, I, —CN, —NO₂, —CF₃, —CN, —NO₂, —CF₃, —OR₄, —SR₄, —S(O)₂R₄, —S(O)R₄, —OS(O)₂R₄, —N(R₄)₂, —C(O)R₄, —C(S)R₄, —C(O)OR₄, —C(O)N(R₄)₂, —N(R₄)C(O)R₄, —N(R₄)C(O)N(R₄)₂, —S(O)₂N(R₄)₂, —N(R₄)S(O)₂R₄;

Lower alkyl is both straight- and branched-chain moieties having from 1-4 carbon atoms;

Lower substituted alkyl is lower alkyl having 1-3 substituents independently selected from F, Cl, Br, or I and further optionally having 1 substituent selected from —CN, —NO₂, —CF₃, —OR₄, —SR₄—S(O)₂R₄, —S(O)R₄, —OS(O)₂R₄, —N(R₄)₂, —C(O)R₄, —(S)R₄, —C(O)OR₄, —C(O)N(R₄)₂, —N(R₄)C(O)R₄, —N(R₄)C(O)N(R₄)₂, —S(O)₂N(R₄)₂, —N(R₄)S(O)₂R₄, or phenyl, wherein phenyl is optionally substituted with up to 4 substituents independently selected from F, Cl, Br, I, —CN, —NO₂, —CF₃, —CN, —NO₂, —CF₃, —OR₄, —SR₄, —S(O)₂R₄, —S(O)R₄, —OS(O)₂R₄, —N(R₄)₂, —C(O)R₄, —C(S)R₄, —C(O)OR₄, —C(O)N(R₄)₂, —N(R₄)C(O)R₄, —N(R₄)C(O)N(R₄)₂, —S(O)₂N(R₄)₂, —N(R₄)S(O)₂R₄;

Alkynyl is straight- and branched-chained moieties having from 2-4 carbon atoms and having at least one carbon-carbon triple bond;

Lower alkynyl is straight- and branched-chained moieties having from 2-3 carbon atoms and having at least one carbon-carbon triple bond;

Each R₄ is independently H or alkyl optionally substituted where valency allows with up to 3 substituents independently selected from —OH, —CN, NH₂, —NO₂, —CF₃, F, Cl, Br, or I;

and pharmaceutically acceptable salts thereof.

The compounds of the present invention are useful to treat, or prepapre a medicament to treat, any one or more of the following: schizophrenia, psychosis, cognitive and attention deficit symptoms of Alzheimer's, neurodegeneration associated with diseases such as Alzheimer's disease, pre-senile dementia (also known as mild cognitive impairment), senile dementia, traumatic brain injury, behavioral and cognitive problems associated with brain tumors, Parkinson's disease, amyotrophic lateral sclerosis, AIDS dementia complex, dementia associated with Down's syndrome, dementia associated with Lewy Bodies, Huntington's disease, attention deficit disorders, attention deficit hyperactivity disorder also known as hyperkinetic disorder, depression, anxiety, general anxiety disorder, post traumatic stress disorder, mood and affective disorders including disruptive and oppositional conditions, borderline personality disorder, panic disorder, tardive dyskinesia, restless leg disorder, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy (e.g., glaucoma and diabetic rentinopathy), symptoms associated with pain (central and peripheral), chemotherapy-induced emesis, migraine, fibromyalgia, irritable bowel syndrome, and diarrhea associated with carcinoid syndrome.

The present invention also includes the compounds of the present invention, pharmaceutical compositions containing the active compounds, and methods to treat the identified diseases.

Abbreviations which are well known to one of ordinary skill in the art may be used (e.g., “Ph” for phenyl, “Me” for methyl, “Et” for ethyl, “h” for hour or hours, “rt” or “RT” for room temperature, and min for minute or minutes).

All temperatures are in degrees Centigrade.

Room temperature is within the range of 15-25 degrees Celsius.

Eq refers to equivalents.

AChR refers to acetylcholine receptor.

nAChR refers to nicotinic acetylcholine receptor.

Pre-senile dementia is also known as mild cognitive impairment.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to dimethylformamide.

EtOAc refers to ethyl acetate.

TMS refers to tetramethylsilane.

TEA refers to triethylamine.

DIEA refers to diisopropylethylamine.

MLA refers to methyllycaconitine.

Ether refers to diethyl ether.

MgSO₄ refers magnesium sulfate.

NaHCO₃ refers to sodium bicarbonate.

KHCO₃ refers to potassium bicarbonate.

CH₃CN refers to acetonitrile.

HATU refers to O-(7-azabenzotriazol-1-yl)-N,N,N′, N′-tetramethyluronium hexafluorophosphate.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Halogen is F, Cl, Br, or I. Halo and halogen are used interchangeably.

Mammal denotes human and other mammals.

Brine refers to an aqueous saturated sodium chloride solution.

IR refers to infrared spectroscopy.

Lv refers to leaving groups within a molecule, including Cl, OH, or mixed anhydride.

Amino protecting group includes, but is not limited to, carbobenzyloxy (CBz), tert butoxy carbonyl (BOC) and the like. Examples of other suitable amino protecting groups are known to person skilled in the art and can be found in “Protective Groups in Organic synthesis,” 3rd Edition, authored by Theodora Greene and Peter Wuts.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

Compounds of the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired effect. The amount of therapeutically effective compound(s) that is administered and the dosage regimen for treating a disease condition with the compounds and/or compositions of this invention depends on a variety of factors, including the age, weight, sex and medical condition of the subject, the severity of the disease, the route and frequency of administration, and the particular compound(s) employed, and thus may vary widely. Thus, it is not possible to specify an exact “effective amount.” However, an appropriate effective amount may be determined by one of ordinary skill in the art using only routine experimentation. The compositions contain well know carriers and excipients in addition to a therapeutically effective amount of compounds of the present invention.

The present invention also includes a pharmaceutical composition comprising a compound of Formula I or a pharmaceutically acceptable salt thereof and a pharmaceutically acceptable excipient. The pharmaceutical composition is administered rectally, topically, orally, sublingually, or parenterally for a therapeutically effective interval. The pharmaceutical composition is administered to deliver a compound of the present invention in an amount of from about 0.001 to about 100 mg/kg of body weight of said mammal per day. The pharmaceutical composition is also administered to deliver a compound of the present invention in an amount of from about 0.1 to about 50 mg/kg of body weight of said mammal per day, or any range therein, e.g., from about 0.1 to about 20 mg/kg of body weight of said mammal per day. The daily dose can be administered in 1-4 doses per day.

A pharmaceutical composition can also comprise a compound of Formula I or a pharmaceutically acceptable salt thereof, an anti-psychotic agent, and a pharmaceutically acceptable excipient. The pharmaceutical composition is administered to independently administer said compound and said agent rectally, topically, orally, sublingually, or parenterally for a therapeutically effective interval. The pharmaceutical composition is administered to deliver a compound of the present invention in an amount of from about 0.001 to about 100 mg/kg of body weight of said mammal per day. The pharmaceutical composition is also administered to deliver a compound of the present invention in an amount of from about 0.1 to about 50 mg/kg of body weight of said mammal per day, or any range therein, e.g., from about 0.1 to about 20 mg/kg of body weight of said mammal per day. The daily dose can be administered in 1-4 doses per day.

In addition to the compound(s) of Formula I, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, e.g., parenterally, bucal, intravaginal, and rectal, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a large degree of sequence homology with α7 nAChR but functionally the two ligand-gated ion channels are very distinct. For example, α7 nAChR is rapidly inactivated, is highly permeable to calcium and is activated by acetylcholine and nicotine. On the other hand, 5HT₃R is inactivated slowly, is relatively impermeable to calcium and is activated by serotonin. These experiments suggest that the α7 nAChR and 5HT₃R proteins have some degree of homology, but function very differently. Indeed the pharmacology of the channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R.

α7 nAChR is a ligand-gated Ca⁺⁺ channel formed by a homopentamer of α7 subunits. Previous studies have established that α-bungarotoxin (α-btx) binds selectively to this homopetameric, α7 nAChR subtype, and that α7 nAChR has a high affinity binding site for both α-btx and methyllycaconitine (MLA). α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and attention-deficit disorders (Potter, A. et. al., Psychopharmacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

The availability of radiolabelled antagonist allowed direct demonstration of central 5-HT₃ receptors (Kilpatrick, et al., 1987; Nature, 330, 746-748). Autoradiographic studies in human brain tissue indicated 5-HT₃ binding sites in forebrain structures and in the medulla oblongata are localized in essentially the same structures as that observed in rat studies. Within the hippocampus, specific binding is restricted to the molecular and granular layers of the dentate gyrus and the pyramidal layer of the CA1, CA2 and CA3 subfields of Ammon's horn. Some specific binding was also found in the amygdala and the entorhinal cortex, whereas the basal ganglia, neocortex, thalamus, cerebellum and the pons were apparently devoid of these receptors (Waeber, et al., 1989; Neuroscience, 31, 393-400; Parker et al, 1996; J. Neurol Sci, 144, 119-127). The limbic location of these receptors is consistent with the notion of regulation of mood, emotion and cognitive functions in man, while the receptors in the brain stem confers the anti-emetic action of these compounds. Binding sites are also detected in the superficial layers of the dorsal horn offering opportunity for control of neuropeptide release and activation of GABAergic pathway to regulation pain transmission.

At regions where α7 and 5-HT₃ receptors are co-localized, for example, at forebrain areas likes hippocampus, striatum, accumbens, hypothalamus, compounds being both α7 agonists and 5-HT₃ antagonists offer a unique blend of regulation of the acetylcholine, dopamine, 5-HT, norepinephrine and growth factor activity that give rise to therapeutic utilities. Said compounds are useful for treating one, or more, or combination of any many diseases or conditions of the central nervous system, including, but not limited to, schizophrenia, psychosis, cognitive and attention deficit symptoms of Alzheimer's, neurodegeneration associated with diseases such as Alzheimer's disease, pre-senile dementia (also known as mild cognitive impairment), senile dementia, traumatic brain injury, behavioral and cognitive problems associated with brain tumors, Parkinson's disease, amyotrophic lateral sclerosis, AIDS dementia complex, dementia associated with Down's syndrome, dementia associated with Lewy Bodies, Huntington's disease, attention deficit disorders, attention deficit hyperactivity disorder also known as hyperkinetic disorder, depression, anxiety, general anxiety disorder, post traumatic stress disorder, mood and affective disorders including disruptive and oppositional conditions, borderline personality disorder, panic disorder, tardive dyskinesia, restless leg disorder, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy (e.g., glaucoma and diabetic rentinopathy), symptoms associated with pain (central and peripheral), chemotherapy-induced emesis, migraine, fibromyalgia, irritable bowel syndrome, and diarrhea associated with carcinoid syndrome.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

Schizophrenia is a complex multifactorial illness caused by genetic and non-genetic risk factors that produce a constellation of positive and negative symptoms. The positive symptoms include delusions and hallucinations and the negative symptoms include deficits in affect, attention, cognition and information processing. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating both the positive and some of the negative symptoms of this disease. Clozapine's utility as a drug is greatly limited because continued use leads to an increased risk of agranulocytosis and seizure. A new generation atypical antipsychotic agent is shown to retain some of the therapeutic advantages of clozapine with reduced toxicity, but show varying degrees of weight gain. No other antipsychotic drug is effective in treating the negative symptoms of schizophrenia. This is significant because the restoration of cognitive functioning is the best predictor of a successful clinical and functional outcome of schizophrenic patients (Green, M. F., Am J Psychiatry, 153:321-30, 1996). By extension, it is clear that better drugs are needed to treat the cognitive disorders of schizophrenia in order to restore a better state of mental health to patients with this disorder.

One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E; et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics.

The compounds of the present invention are α7 nAChR agonists and may be used to treat a wide variety of diseases. For example, they may be used in treating schizophrenia, or psychosis.

Schizophrenia is a disease having multiple aspects. Currently available drugs are generally aimed at controlling the positive aspects of schizophrenia, such as delusions. One drug, Clozapine, is aimed at a broader spectrum of symptoms associated with schizophrenia. This drug has many side effects and is thus not suitable for many patients. Thus, there is a need for a drug to treat the cognitive and attention deficits associated with schizophrenia. Similarly, there is a need for a drug to treat the cognitive and attention deficits associated with schizoaffective disorders, or similar symptoms found in the relatives of schizophrenic patients.

Psychosis is a mental disorder characterized by gross impairment in the patient's perception of reality. The patient may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

There are a variety of antipsychotic drugs. The conventional antipsychotic drugs include Chlorpromazine, Fluphenazine, Haloperidol, Loxapine, Mesoridazine, Molindone, Perphenazine, Pimozide, Thioridazine, Thiothixene, and Trifluoperazine. These drugs all have an affinity for the dopamine 2 receptor.

These conventional antipsychotic drugs have several side effects, including sedation, weight gain, tremors, elevated prolactin levels, akathisia (motor restlessness), dystonia and muscle stiffness. These drugs may also cause tardive dyskinesia. Unfortunately, only about 70% of patients with schizophrenia respond to conventional antipsychotic drugs. For these patients, atypical antipsychotic drugs are available.

Atypical antipsychotic drugs generally are able to alleviate positive symptoms of psychosis while also improving negative symptoms of the psychosis to a greater degree than conventional antipsychotics. These drugs may improve neurocognitive deficits. Extrapyramidal (motor) side effects are not as likely to occur with the atypical antipsychotic drugs, and thus, these atypical antipsychotic drugs have a lower risk of producing tardive dyskinesia. Finally these atypical antipsychotic drugs cause little or no elevation of prolactin. Unfortunately, these drugs are not free of side effects. Although these drugs each produce different side effects, as a group the side effects include: agranulocytosis; increased risk of seizures, weight gain, somnolence, dizziness, tachycardia, decreased ejaculatory volume, and mild prolongation of QTc interval.

In a combination therapy to treat multiple symptoms of diseases such as schizophrenia, the compounds of Formula I and the anti-psychotic drugs (typical and atypical) can be administered simultaneously or at separate intervals. When administered simultaneously the compounds of Formula I and the anti-psychotic drugs can be incorporated into a single pharmaceutical composition, e.g., a pharmaceutical combination therapy composition. Alternatively, two separate compositions, i.e., one containing compounds of Formula I and the other containing anti-psychotic drugs, can be administered simultaneously. Examples of anti-psychotic drugs, in addition to those listed above, include, but are not limited to, Thorazine, Mellaril, Trilafon, Navane, Stelazine, Permitil, Prolixin, Risperdal, Zyprexa, Seroquel, Zeldox, Acetophenazine, Carphenazine, Chlorprothixene, Droperidol, Loxapine, Mesoridazine, Molindone, Ondansetron, Pimozide, Prochlorperazine, Promazine, Geodon, Quietipine, and Aripreparol.

A pharmaceutical combination therapy composition can include therapeutically effective amounts of the compounds of Formula I, noted above, and a therapeutically effective amount of anti-psychotic drugs. These compositions may be formulated with common excipients, diluents or carriers, and compressed into tablets, or formulated elixirs or solutions for convenient oral administration or administered by intramuscular or intravenous routes. The compounds can be administered rectally, topically, orally, sublingually, or parenterally and maybe formulated as sustained relief dosage forms and the like.

When separately administered, therapeutically effective amounts of compositions containing compounds of Formula I and anti-psychotic drugs are administered on a different schedule. One may be administered before the other as long as the time between the two administrations falls within a therapeutically effective interval. A therapeutically effective interval is a period of time beginning when one of either (a) the compounds of Formula I, or (b) the anti-psychotic drugs is administered to a human and ending at the limit of the beneficial effect in the treatment of schizophrenia or psychosis of the combination of (a) and (b). The methods of administration of the compounds of Formula I and the anti-psychotic drugs may vary. Thus, either agent or both agents may be administered rectally, topically, orally, sublingually, or parenterally.

As discussed, the compounds of the present invention are α7 nAChR agonists and 5-HT₃ antagonists. Therefore, as another aspect of the present invention, the compounds of the present invention may be used to treat a variety of diseases including cognitive and attention deficit symptoms of Alzheimer's, neurodegeneration associated with diseases such as Alzheimer's disease, pre-senile dementia (also known as mild cognitive impairment), senile dementia, traumatic brain injury, behavioral and cognitive problems associated with brain tumors, or Parkinson's disease.

Alzheimer's disease has many aspects, including cognitive and attention deficits. Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for a drug that stimulates a portion of the cholinergic pathways and thereby provides improvement in the cognitive and attention deficits associated with Alzheimer's disease without the side effects created by nonspecific stimulation of the cholinergic pathways.

Neurodegeneration is a common problem associated with diseases such as Alzheimer's disease. While the current drugs treat some of the symptoms of this disease, they do not control the underlying pathology of the disease. Accordingly, it would be desirable to provide a drug that can slow the progress of Alzheimer's disease.

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

Parkinson's disease is a neurological disorder characterized by tremor, hypokinesia, and muscular rigidity. Currently, there is no treatment to stop the progression of the disease. Therefore, there is a need of a pharmaceutical agent to address Parkinson's.

As discussed, the compounds of the present invention are α7 nAChR agonists and 5-HT₃ antagonists. Therefore, yet other diseases to be treated with compounds of the present invention include treating amyotrophic lateral sclerosis, AIDS dementia complex, dementia associated with Down's syndrome, dementia associated with Lewy Bodies, Huntington's disease, attention deficit disorders, attention deficit hyperactivity disorder, depression, anxiety, general anxiety disorder, post traumatic stress disorder, mood and affective disorders including disruptive and oppositional conditions, borderline personality disorder, panic disorder, tardive dyskinesia, restless leg syndrome, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy (e.g., glaucoma and diabetic rentinopathy), symptoms associated with pain (central and peripheral), chemotherapy-induced emesis, migraine, fibromyalgia, irritable bowel syndrome, and diarrhea associated with carcinoid syndrome.

Amyotrophic lateral sclerosis, also known as Lou Gehrig's disease, belongs to a class of disorders known as motor neuron diseases wherein specific nerve cells in the brain and spinal cord gradually degenerate to negatively affect the control of voluntary movement. Currently, there is no cure for amyotrophic lateral sclerosis although patients may receive treatment from some of their symptoms and although Riluzole has been shown to prolong the survival of patients. Therefore, there is a need for a pharmaceutical agent to treat this disease.

Acquired immune deficiency syndrome (AIDS) results from an infection with the human immunodeficiency virus (HIV). This virus attacks selected cells and impairs the proper function of the immune, nervous, and other systems. HIV infection can cause other problems such as, but not limited to, difficulties in thinking, otherwise known as AIDS dementia complex. Therefore, there is a need to drugs to relieve the confusion and mental decline of persons with AIDS.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Dementia with Lewy Bodies is a neurodegenerative disorder involving abnormal structures known as Lewy bodies found in certain areas of the brain. Symptoms of dementia with Lewy bodies include, but are not limited to, fluctuating cognitive impairment with episodic delirium. Currently, treatment concerns addressing the parkinsonian and psychiatric symptoms. However, medicine to control tremors or loss of muscle movement may actually accentuate the underlying disease of dementia with Lewy bodies. Therefore, there is a need of a pharmaceutical agent to treat dementia with Lewy bodies.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

Attention deficit disorder is generally treated with methylphenidate, an amphetamine-like molecule that has some potential for abuse. Accordingly, it would be desirable to provide a drug that treats attention deficit disorder while having fewer side effects than the currently used drug.

Attention deficit hyperactivity disorder (ADHD) also known as hyperkinetic disorder, is a neurobehavioral disorder affecting 3-5% of all American children. ADHD concerns cognitive alone or both cognitive and behavioral actions by interfering with a person's ability to stay on a task and to exercise age-appropriate inhibition. Several types of ADHD exist: a predominantly inattentive subtype, a predominantly hyperactive-impulsive subtype, and a combined subtype. Treatment may include medications such as methylphenidate, dextroamphetamine, or pemoline, which act to decrease impulsivity and hyperactivity and to increase attention. No “cure” for ADHD currently exists. Children with the disorder seldom outgrow it; therefore, there is a need for appropriate medicaments.

Depression is a mood disorder affecting 10% of the general population, manifesting of varying lengths of ranging from several months to more than two years and of varying degrees of feelings involving sadness, despair, and discouragement. The heterocyclic antidepressants (HCA's) are currently the largest class of antidepressants, but monoamine oxidase inhibitors (MAOI's) are used in particular types of depression. Common side effects from HCA's are sedation, dry mount, sexual dysfunction, and weight gain. In elderly patients with organic brain disease, the side effects from HCA's can also include seizures and behavioral symptoms. The main side effects from using MAOI's occur from dietary and drug interactions. The alternative to the above therapy is electronic convulsion therapy having a side effect of memory loss. Therefore, agents with fewer side effects would be helpful.

Anxiety disorders (disorders with prominent anxiety or phobic avoidance), represent an area of umet medical needs in the treatment of psychiatric illness. See Diagnostic & Statistical Manual of Mental Disorders, IV (1994), pp 393-394, for various disease forms of anxiety.

General anxiety disorder (GAD) occurs when a person worries about things such as family, health, or work when there is no reason to worry and is unable not to worry. About 3 to 4% of the U.S. population has GAD during the course of a year. GAD most often strikes people in childhood or adolescence, but can begin in adulthood, too. It affects women more often than men. Currently, treatment involves cognitive-behavioral therapy, relaxation techniques, and biofeedback to control muscle tension and medications such as benzodiazepines, imipramine, and buspirone. These drugs are effective but all have side-effect liabilities. Therefore, there is a need of a pharmaceutical agent to address the symptoms with fewer side effects.

Anxiety also includes post-traumatic stress disorder (PTSD), which is a form of anxiety triggered by memories of a traumatic event that directly affected the patient or that the patient may have witnessed. The disorder commonly affects survivors of traumatic events including sexual assault, physical assault, war, torture, natural disasters, an automobile accident, an airplane crash, a hostage situation, or a death camp. The affliction also can affect rescue workers at an airplane crash or a mass shooting, someone who witnessed a tragic accident or someone who has unexpectedly lost a loved one. Treatment for PTSD includes cognitive-behavioral therapy, group psychotherapy, and medications such as Clonazepam, Lorazepam and selective serotonin-reuptake inhibitors such as Fluoxetine, Sertraline, Paroxetine, Citalopram and Fluvoxamine. These medications help control anxiety as well as depression. Various forms of exposure therapy (such as systemic desensitization and imaginal flooding) have all been used with PTSD patients. Exposure treatment for PTSD involves repeated reliving of the trauma, under controlled conditions, with the aim of facilitating the processing of the trauma. Therefore, there is a need for better pharmaceutical agents to treat post traumatic stress disorder.

Mood and affective disorders fall within a large group of diseases, including monopolar depression and bi-polar mood disorder. These diseases are treated with three major classes of compounds. The first group is the heterocyclic antidepressant (HCA's). This group includes the well-known tricyclic antidepressants. The second group of compounds used to treat mood disorders is the monoamine oxidase inhibitors (MAOI's) that are used in particular types of diseases. The third drug is lithium. Common side effects from HCA's are sedation and weight gain. In elderly patients with organic brain disease, the side effects of HCA's can also include seizures and behavioral symptoms. The main side effects from using MAOI's occur from dietary and drug interactions. Benign side effects from the use of lithium include, but are not limited to, weight gain, nausea, diarrhea, polyuria, polydipsia, and tremor. Toxic side effects from lithium can include persistent headache, mental confusion, and may reach seizures and cardiac arrhythmias. Therefore, agents with less side effects or interactions with food or other medications would be useful.

Borderline personality disorder, although not as well known as bipolar disorder, is more common. People having borderline personality disorder suffer from a disorder of emotion regulation. Pharmaceutical agents are used to treat specific symptoms, such as depression or thinking distortions.

Panic is the acute, sudden and intense form of anxiety. A panic attack is defined as a discrete period of intense fear or discomfort accompanied by somatic and cognitive symptoms. The anxiety that is characteristic of a panic attack can be differentiated from generalized anxiety by its intermittent, almost paroxysmal nature and its typically greater severity. Panic disorder is characterized by recurrent panic attacks, anticipatory anxiety, agoraphobia, hypochondriasis and demoralization/secondary depression. Schlegal and colleagues (1994; Eur Arch Psychia Clin Neuorsci, 244, 49-51) were the first to report a decreased of GABAergic activity in panic disorder using lomazenil SPECT. The decreases were significant in the occipital and frontral cortices and maximal in the temporal cortex. This invention concerns the dual action of the said molecules would synergize to reduce the anxiety by 5-HT3 receptor antagonism and increase GABAergic tone by alpha7 nicotinic receptor activation.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of tardive dyskinesia.

Restless leg syndrome (RLS) is a neurosensorimotor disorder with parestethesias, sleep disturbances and, in most cases, periodic limb movements of sleep (PLMS). Treatment of RLS and PLMS has varied and includes clonazepam and other benzodiazepines, propoxyphene and other opiates, and L-dopa and other dopoaminergic drugs. While L-dopa has been used somewhat successfully in the treatment of PLMS, often-repeated dosages over the course of the night are required. Dosages effective in the treatment of PLMS also can lead to daytime drowsiness in some patients. The sustained-release form of carbidopa-levodopa was thought to be the answer to repeated nighttime dosages; however, this has not been borne out in clinical studies. Therefore, there is a need to effectively treat patients afflicted with RLS and PLMS.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions hallucinations, and narcotics. Therefore, there is a need for a pharmaceutical agent to treat the progressive deterioration of social skills and changes in personality and to address the symptoms with fewer side effects.

Dysregulation of food intake associated with eating disease, including bulemia nervosa and anorexia nervosa, involve neurophysiological pathways. Anorexia nervosa is hard to treat due to patients not entering or remaining in after entering programs. Currently, there is no effective treatment for persons suffering from severe anorexia nervosa. Cognitive behavioral therapy has helped patients suffering from bulemia nervosa; however, the response rate is only about 50% and current treatment does not adequately address emotional regulation. Therefore, there is a need for pharmaceutical agents to address neurophysiological problems underlying diseases of dysregulation of food intake.

Cigarette smoking has been recognized as a major public health problem for a long time. However, in spite of the public awareness of health hazard, the smoking habit remains extraordinarily persistent and difficult to break. There are many treatment methods available, and yet people continue to smoke. Administration of nicotine transdermally, or in a chewing gum base is common treatments. However, nicotine has a large number of actions in the body, and thus can have many side effects. It is clear that there is both a need and a demand of long standing for a convenient and relatively easy method for aiding smokers in reducing or eliminating cigarette consumption. A drug that could selectively stimulate only certain of the nicotinic receptors would be useful in smoke cessation programs.

Smoke cessation programs may involve oral dosing of the drug of choice. The drug may be in the form of tablets. However, it is preferred to administer the daily dose over the waking hours, by administration of a series of incremental doses during the day. The preferred method of such administration is a slowly dissolving lozenge, troche, or chewing gum, in which the drug is dispersed. Another drug in treating nicotine addiction is Zyban. This is not a nicotine replacement, as are the gum and patch. Rather, this works on other areas of the brain, and its effectiveness is to help control nicotine craving or thoughts about cigarette use in people trying to quit. Despite these treatments, more effective drugs are needed to assist smokers in their desire to stop smoking. These drugs may be administered transdermally through the use of skin patches. In certain cases, the drugs may be administered by subcutaneous injection, especially if sustained release formulations are used.

Drug use and dependence is a complex phenomenon, which cannot be encapsulated within a single definition. Different drugs have different effects, and therefore different types of dependence. Drug dependence has two basic causes, that is, tolerance and physical dependence. Tolerance exists when the user must take progressively larger doses to produce the effect originally achieved with smaller doses. Physical dependence exists when the user has developed a state of physiologic adaptation to a drug, and there is a withdrawal (abstinence) syndrome when the drug is no longer taken. A withdrawal syndrome can occur either when the drug is discontinued or when an antagonist displaces the drug from its binding site on cell receptors, thereby counteracting its effect. Drug dependence does not always require physical dependence.

In addition drug dependence often involves psychological dependence, that is, a feeling of pleasure or satisfaction when taking the drug. These feelings lead the user to repeat the drug experience or to avoid the displeasure of being deprived of the drug. Drugs that produce strong physical dependence, such as nicotine, heroin and alcohol are often abused, and the pattern of dependence is difficult to break. Drugs that produce dependence act on the CNS and generally reduce anxiety and tension; produce elation, euphoria, or other pleasurable mood changes; provide the user feelings of increased mental and physical ability; or alter sensory perception in some pleasurable manner. Among the drugs that are commonly abused are ethyl alcohol, opioids, anxiolytics, hypnotics, cannabis (marijuana), cocaine, amphetamines, hallucinogens, and narcotics. The current treatment for drug-addicted people often involves a combination of behavioral therapies and medications. Medications, such as methadone or LAAM (levo-alpha-acetyl-methadol), are effective in suppressing the withdrawal symptoms and drug craving associated with narcotic addiction, thus reducing illicit drug use and improving the chances of the individual remaining in treatment. The primary medically assisted withdrawal method for narcotic addiction is to switch the patient to a comparable drug that produces milder withdrawal symptoms, and then gradually taper off the substitute medication. The medication used most often is methadone, taken by mouth once a day. Patients are started on the lowest dose that prevents the more severe signs of withdrawal and then the dose is gradually reduced. Substitutes can be used also for withdrawal from sedatives. Patients can be switched to long-acting sedatives, such as diazepam or phenobarbital, which are then gradually reduced.

Gilles de la Tourette's Syndrome is an inherited neurological disorder. The disorder is characterized by uncontrollable vocal sounds called tics and involuntary movements. The symptoms generally manifest in an individual before the person is 18 years of age. The movement disorder may begin with simple tics that progress to multiple complex tics, including respiratory and vocal ones. Vocal tics may begin as grunting or barking noises and evolve into compulsive utterances. Coprolalia (involuntary scatologic utterances) occurs in 50% of patients. Severe tics and coprolalia may be physically and socially disabling. Tics tend to be more complex than myoclonus, but less flowing than choreic movements, from which they must be differentiated. The patient may voluntarily suppress them for seconds or minutes.

Currently simple tics are often treated with benzodiazepines. For simple and complex tics, Clonidine may be used. Long-term use of Clonidine does not cause tardive dyskinesia; its limiting adverse effect is hypotension. In more severe cases, antipsychotics, such as Haloperidol may be required, but side effects of dysphoria, parkinsonism, akathisia, and tardive dyskinesia may limit use of such antipsychotics. There is a need for a safe and effective methods for treating this syndrome.

Age-related macular degeneration (AMD) is a common eye disease of the macula which is a tiny area in the retina that helps produce sharp, central vision required for “straight ahead” activities that include reading and driving. Persons with AMD lose their clear, central vision. AMD takes two forms: wet and dry. In dry AMD, there is a slow breakdown of light-sensing cells in the macula. There currently is no cure for dry AMD. In wet AMD, new, fragile blood vessels growing beneath the macula as dry AMD worsens and these vessels often leak blood and fluid to cause rapid damage to the macula quickly leading to the loss of central vision. Laser surgery can treat some cases of wet AMD. Therefore, there is a need of a pharmaceutical agent to address AMD.

Glaucoma is within a group of diseases that occurs from an increase in intraocular pressure causing pathological changes in the optical disk and optic nerve, and negatively affects the field of vision. Medicaments to treat glaucoma either decrease the amount of fluid entering the eye or increase drainage of fluids from the eye in order to decrease intraocular pressure. However, current drugs have drawbacks such as not working over time or causing side effects so the eye-care professional has to either prescribe other drugs or modify the prescription of the drug being used. Furthermore, a significant number of glaucoma patients exhibit disease progression while having normal IOP. There is a need for safe and effective methods for treating problems manifesting into glaucoma.

Ischemic periods in glaucoma cause release of excitotoxic amino acids and stimulate inducible form of nitric oxide synthase (iNOS) leading to neurodegeneration. Alpha 7 nicotinic agonists may stimulate the release of inhibitory amino acids such as GABA which will dampen hyperexcitablity. Alpha 7 nicotinic agonists are also directly neuroprotective on neuronal cell bodies. Thus alpha 7 nicotinic agonists have the potential to be neuroprotective in glaucoma.

The physiological role of 5-HT as a message in the ocular system is implicated by the demonstration of the serotonin receptors and transporters in mammalian retina (Brunken and Jin, 1993; Visual Neuroscience, 10, 511-522). 5-HT₃ receptors in the mammalian receptors have been reported to mediate excitatory influence in the retina (Brunken et al, 1993; Prog. Retinal Res., 12, 75-99). Therefore, compounds being both a 5-HT₃ antagonist and an α7 agonist would dampen hyperexcitability.

Diabetic retinopathy is the most common complication of diabetes, affecting over 90% of persons with diabetes and progressing to legal blindness in about 5%. The vascular features of long-term diabetic retinopathy are well documented, but non-vascular pathology has received less attention until a recent observation that both experimental diabetes in rats and diabetes mellitus in humans are accompanied by increased apoptosis of retinal neural cells (Barber et al, 1998; J Clin Invest, 102, 783-791). The increase in the frequency of apoptosis occurred after only 1 month of experimental diabetes in rats is similar to that observed in a human retina after 6 years of diabetes. The significant reduction of retinal ganglion cells and the reduction in the thickness of the inner plexiform and nuclear layers after 7.5 months of streptozocin (STZ) induced diabetes suggest that the apoptotic cells include ganglion cells and other neurons. Therefore, neurodegeneration could be an important feature of diabetic retinopathy (Bloodworth, 1962; Diabetes, 2, 1-22). The value of considering α7 receptor mediated neuroprotection in this context is the ability to increase neurotrophic factor influence in cellular population in the retina to reduce their vulnerability in response to the metabolic and other diabetic related insults. Blockade of the 5-HT receptor might dampen hyperexcitability.

Persons afflicted with pain often have what is referred to as the “terrible triad” of suffering from the pain, resulting in sleeplessness and sadness, all of which are hard on the afflicted individual and that individual's family. Pain can manifest itself in various forms, including, but not limited to, headaches of all severity, back pain, neurogenic, and pain from other ailments such as arthritis and cancer from its existence or from therapy to irradicate it. Pain can be either chronic (persistent pain for months or years) or acute (short-lived, immediate pain to inform the person of possible injury and need of treatment. Persons suffering from pain respond differently to individual therapies with varying degrees of success. There is a need for a safe and effective methods for treating pain.

The highest density of 5-HT₃ receptors in the CNS are found in the brain medulla oblongata, in four key regions namely the nucleus tractus solitarius (NTS), the dorsal motor nucleus of the vagus nerve, the area postrema, and the nucleus of the spinal tract of the trigeminal nerve (Kilpatrick, et al., 1990; Medicinal Res., 10, 441-475). Local injection of 5-HT₃ antagonists into the area postrema and NTS provide the anatomical support for their potent effects in preventing nausea and emesis due to cytotoxic drugs in vomiting (Higgins, et al., 1989, Br. J. Pharmacol., 97, 247-25; Perez, et al., 1991, Seminars Oncol., 18, 73-80). While the emesis component of cancer chemotherapy is being managed by 5-HT₃ antagonists in the market, the cytotoxic drugs continue to exert their toxic influence on all cells of the body, including neurons in the CNS. A molecule with dual action as a 5-HT₃ receptor antagonist and alpha7 nicotinic receptor agonist has the novel feature of providing neuroprotection influence via alpha 7 action while maintaining anti-emetic efficacy. Likewise, these molecules are expected to be exceptional for the control of neuronal hyperexcitability and nausea associated with migraine (Ferrari, 1991; J Neurol, 238, 553-556), and the prophylactic treatment of migraine.

Fibromyalgia by definition represents an inflammation of the fibrous tissues of the muscles, fascia, aponeuroses, and probably nerves as well, leading to pain and tenderness of a muscle or diffuse across the skeletal system, particular after exposure to cold, dampness, or minor trauma, but often for no reason as all. So far, the pathologic basis of this state remains unclear. Given the role of 5-HT₃ receptors in the brain stem regulating neurovegatative function, and pain transmission in the spinal cord, 5-HT₃ receptor antagonists, in particular tropisetron, have been shown to decrease tenderness at “tenderpoints” and reduction in pain-score (Farber, et al., 2001; Int. J. Clin. Pharmacol. Res., 21, 1-13).

5-HT₃ receptor activation results in cholinergic and non-cholinergic transmission, producing contractile response and fluid secretion in the GI tract (Cohen, et al., 1985, J. Pharmacol. Exp. Ther., 232, 770-774; Boeckxstaens, et al., 1990, J. Pharmacol. Exp. Ther., 254, 652-658). Given the roles these receptors play in colonic sensory and motor function, 5-HT₃ receptor antagonists have been proposed for the treatment of irritable bowel syndrome (Camilleri, et al., 1999; Aliment Pharmacol. Ther., 13, 1149-59) and diarrhea associated with carcinoid sydrome (Anderson, et al., 1987; Br. Med. J., 294, 1129). The advantages of a molecule with dual activity as a 5-HT₃ receptor antagonist and an alpha 7 agonist is the additional feature of handling pain mediating neurodegeneration.

Finally, the compounds of the present invention may be used in combination therapy with typical and atypical anti-psychotic drugs. All compounds within the present invention are useful for and may also be used in combination with each other to prepare pharmaceutical compositions. Such combination therapy lowers the effective dose of the anti-psychotic drug and thereby reduces the side effects of the anti-psychotic drugs. Some typical anti-psychotic drugs that may be used in the practice of the invention include Haldol. Some atypical anti-psychotic drugs include Ziprasidone, Olanzapine, Resperidone, and Quetiapine.

Compounds of Formula I can be prepared as shown in Scheme 1. The key step in the preparation of this class of compounds is the coupling of an amino-azabicyclic moiety with the requisite acid chloride (Lv=Cl), mixed anhydride (e.g., Lv is diphenyl phosphoryl, bis(2-oxo-3-oxazolidinyl)phosphinyl, or acyloxy of the general formula of O—C(O)—R_{Lv}, where R_{Lv} includes phenyl or t-butyl), or carboxylic acid (Lv is OH) in the presence of an activating agent. Suitable activating reagents are well known in the art, for examples see Kiso, Y., Yajima, H. “Peptides” pp. 39-91, San Diego, Calif., Academic Press, (1995), and include, but are not limited to, agents such as carbodiimides, phosphonium and uronium salts (such as HATU).

### Scheme 1

Lv—C(═O)—W⁰+H₂N-Azabicyclo→W⁰—N(H)-Azabicyclo

Generally, the acid is activated using HATU or is converted to the acyl azide by using DPPA or is converted into a mixed anhydride by treatment with bis (2-oxo-3-oxazolidinyl) phosphinic chloride in the presence of TEA with CH₂Cl₂ or CHCl₃ as the solvent. In the case where R₃ is tert-butyloxycarbonyl (where Azabicyclo is III), deprotection of the 7-aza group can be conveniently accomplished under acidic conditions in a suitable solvent such as methanol.

The appropriate amine is reacted with TEA if the amine is in the form of an acid salt and added to a solution of the appropriate anhydride or azide to give the desired final compounds. In some cases, the ester (Lv being OMe or OEt) may be reacted directly with the amine in refluxing methanol or ethanol to give the compounds of Formula I.

One of ordinary skill in the art will recognize that the methods described for the reaction of the unsubstituted 3-aminoquinuclidine (R₂=H) are equally applicable to substituted compounds (R₂≠H). Such compounds can be prepared by reduction of the oxime of the corresponding 3-quinuclidinone (see J. Labelled Compds. Radiopharm., 53-60 (1995) and J. Med. Chem. 988-995, (1998)). The oximes can be prepared by treatment of the 3-quinuclidinones with hydroxylamine hydrochloride in the presence of a base. The 3-quinuclidinones, where R₂=substituted alkyl, or cycloalkyl can be prepared by known procedures (see Tet. Lett. 1015-1018, (1972), J. Am. Chem. Soc. 1278-1291 (1994) J. Am. Chem. Soc. 4548-4552 (1989), Tetrahedron, 1139-1146 (2000)). The 3-quinuclidinones, where R₂=aryl, can be prepared by palladium catalyzed arylation as described in J. Am. Chem. Soc. 1473-1478 (1999) and J. Am. Chem. Soc. 1360-1370 (2000).

One of ordinary skill in the art will recognize that the methods described for the reaction of the unsubstituted 3-amino-1-azabicyclo[2.2.1]heptane (R₂=H) are equally applicable to substituted compounds (R₂≠H). For where Azabicyclo II has substitution at C-2, compounds can be prepared from appropriately substituted nitro alcohols using procedures described in Tetrahedron (1997), 53, p. 11121 as shown below. Methods to synthesize nitro alcohols are well known in the art (see J. Am. Chem. Soc. (1947), 69, p 2608). The scheme below is a modification of the synthesis of exo-3-amino-1-azabicyclo[2.2.1]heptane as the bis(hydro para-toluenesulfonate) salt, described in detail herein, to show how to obtain these amine precursors. The desired salt can be made using standard procedures.

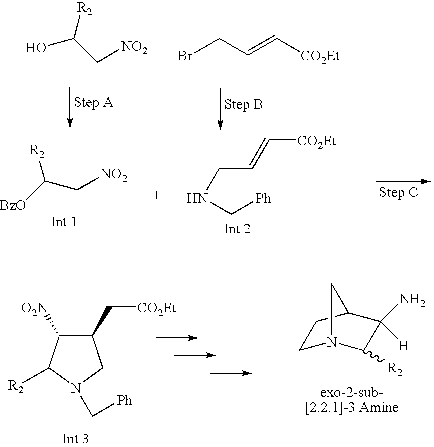

For Azabicyclo II where R₂ is other than H at the C-6 position, compounds can also be prepared by modification of intermediates described in the synthesis of exo-3-amino-1-azabicyclo[2.2.1]heptane as the bis(hydro para-toluenesulfonate) salt, described in detail herein. For example, Int 6 can be oxidized to the aldehyde and treated with an organometallic reagent to provide Int 20 using procedures described in Tetrahedron (1999), 55, p 13899. Int 20 can be converted into the amine using methods described for the synthesis of exo-3-amino-1-azabicyclo[2.2.1]heptane as the bis(hydro para-toluenesulfonate) salt. Once the amine is obtained, the desired salt can be made using standard procedures.

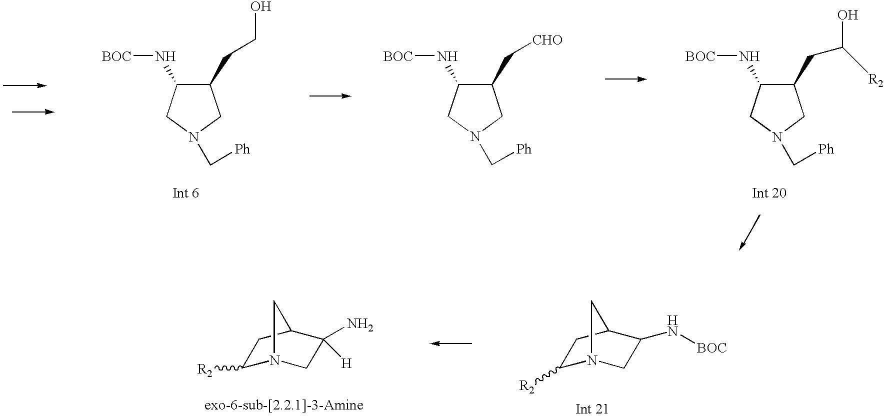

The schemes used are for making exo-3-amino-1-azabicyclo[2.2.1]heptane. However, the modifications discussed are applicable to make the endo isomer also.

### AMINES

Preparation of N-(2S,3R)-2-methyl-1-azabicyclo[2.2.2]octan-3-amine dihydrochloride (2S-methyl-2.2.2-Amine): See, e.g., U.S. 20020042428 A1.

### Preparation of the 1-azabicyclo-2.2.1 Amines

Synthesis of exo-3-amino-1-azabicyclo[2.2.1]heptane as the bis(hydro para-toluenesulfonate) salt (exo-[2.2.1]-Amine):

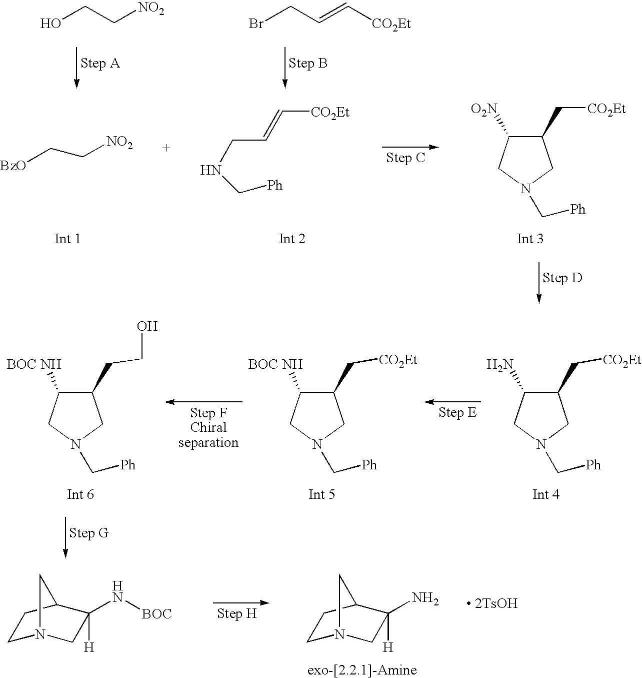

### Step A

### Preparation of 2-(benzoyloxy)-1-nitroethane (Int 1)

Benzoyl chloride (14.9 mL, 128 mmol) is added to a stirred solution of nitroethanol (9.2 mL, 128 mmol) in dry benzene (120 mL). The solution is refluxed for 24 hr and then concentrated in vacuo. The crude product is purified by flash chromatography on silica gel. Elution with hexanes-EtOAc (80:20) affords Int 1 as a white solid (68% yield): ¹H NMR (CDCl₃) δ 8.0, 7.6, 7.4, 4.9, 4.8.

### Step B

### Preparation of ethyl E-4-(benzylamino)-2-butenoate (Int 2)

Ethyl E-4-bromo-2-butenoate (10 mL, 56 mmol, tech grade) is added to a stirred solution of benzylamine (16 mL, 146 mmol) in CH₂Cl₂ (200 mL) at rt. The reaction mixture stirs for 15 min, and is diluted with ether (1 L). The mixture is washed with saturated aqueous NaHCO₃ solution (3×) and water, dried over Na₂SO₄, filtered and concentrated in vacuo. The residue is purified by flash chromatography on silica gel. Elution with hexanes-EtOAc (70:30) affords Int 2 as a clear oil (62% yield): ¹H NMR (CDCl₃) δ 7.4-7.2, 7.0, 6.0, 4.2, 3.8, 3.4, 2.1-1.8, 1.3.

### Step C

### Preparation of trans-4-nitro-1-(phenylmethyl)-3-pyrrolidineacetic acid ethyl ester (Int 3)

A solution of Int 1 (6.81 g, 34.9 mmol) and Int 2 (7.65 g, 34.9 mmol) in EtOH (70 mL) stirs at rt for 15 h and is then concentrated in vacuo. The residue is diluted with ether (100 mL) and saturated aqueous NaHCO₃ solution (100 mL). The organic layer is separated and dried over Na₂SO₄, filtered and concentrated in vacuo. The crude product is purified by flash chromatography on silica gel. Elution with hexanes-EtOAc (85:15) affords Int 3 as a clear oil (76% yield): ¹H NMR (CDCl₃) δ 7.4-7.3, 4.8-4.7, 4.1, 3.8-3.6, 3.3-3.0, 2.7-2.6, 2.4-2.3, 1.2.

### Step D

### Preparation of trans-4-amino-1-(phenylmethyl)-3-pyrrolidineacetic acid ethyl ester (Int 4)

A mixture of Int 3 (3.28 g, 11.2 mmol) and RaNi (1.5 g) in EtOH (100 mL) is placed in a Parr bottle and hydrogenated for 4 h under an atmosphere of hydrogen (46 psi) at rt. The mixture is filtered through a pad of Celite, and the solvent is removed in vacuo to afford Int 4 as a clear oil (100% yield): ¹H NMR (300 MHz, CDCl₃) δ 7.3-7.2, 4.1, 3.6, 3.2, 3.0-2.9, 2.8, 2.8-2.6, 2.6-2.4, 2.30-2.2, 1.2.

### Step E

### Preparation of trans-4-(1,1-dimethylethoxycarbonylamido)-1-(phenylmethyl)-3-pyrrolidineacetic acid ethyl ester (Int 5)

Di-tert-butyldicarbonate (3.67 g, 16.8 mmol) is added to a stirred solution of Int 4 (2.94 g, 11.2 mmol) in CH₂Cl₂ (30 mL) cooled in an ice bath. The reaction is allowed to warm to rt and stirred overnight. The mixture is concentrated in vacuo. The crude product is purified by flash chromatography on silica gel. Elution with hexanes-EtOAc (80:20) affords Int 5 as a white solid (77% yield): ¹H NMR (300 MHz, CDCl₃) δ 7.4-7.2, 5.1-4.9, 4.1, 4.0-3.8, 3.6, 3.2-3.0, 2.8-2.6, 2.5-2.4, 2.3-2.1, 1.4, 1.3.

### Step F

### Preparation of trans (tert-butoxycarbonylamino)-4-(2-hydroxyethyl)-1-(N-phenylmethyl) pyrrolidine (Int 6)

LiAlH₄ powder (627 mg, 16.5 mmol) is added in small portions to a stirred solution of Int 5 (3.0 g, 8.3 mmol) in anhydrous THF (125 mL) in a −5° C. bath. The mixture is stirred for 20 min in a −5° C. bath, then quenched by the sequential addition of water (0.6 mL), 15% (w/v) aqueous NaOH (0.6 mL) and water (1.8 mL). Excess anhydrous K₂CO₃ is added, and the mixture is stirred for 1 h, then filtered. The filtrate is concentrated in vacuo. The residue is purified by flash chromatography on silica gel. Elution with EtOAc affords Int 6 as a white solid (94% yield): ¹H NMR (CDCl₃) δ 7.4-7.3, 5.3-5.2, 4.1-4.0, 3.9-3.7, 3.3-3.2, 2.8-2.7, 2.3-2.1, 1.7, 1.5.

Int 6 is a racemic mixture that can be resolved via chromatography using a Diacel chiral pack AD column. From the two enantiomers thus obtained, the (+)-enantiomer, [α]²⁵_{D}+35 (c 1.0, MeOH), gives rise to the corresponding optically pure exo-4-S final compounds, whereas the (−)-enantiomer, [α]²⁵_{D}−34 (c 0.98, MeOH), gives rise to optically pure exo-4-R final compounds. The methods described herein use the (+)-enantiomer of Int 6 to obtain the optically pure exo-4-S final compounds. However, the methods used are equally applicable to the (−)-enantiomer of Int 6, making non-critical changes to the methods provided herein to obtain the optically pure exo-4-R final compounds.

### Step G

### Preparation of exo 3-(tert-butoxycarbonylamino)-1-azabicyclo[2.2.1]heptane (Int 7)

TEA (8.0 g, 78.9 mml) is added to a stirred solution of Int 6 (2.5 g, 7.8 mmol) in CH₂Cl₂ (50 mL), and the reaction is cooled in an ice-water bath. CH₃SO₂Cl (5.5 g, 47.8 mmol) is then added dropwise, and the mixture is stirred for 10 min in an ice-water bath. The resulting yellow mixture is diluted with saturated aqueous NaHCO₃ solution, extracted with CH₂Cl₂ several times until no product remains in the aqueous layer by TLC. The organic layers are combined, washed with brine, dried over Na₂SO₄ and concentrated in vacuo. The residue is dissolved in EtOH (85 mL) and is heated to reflux for 16 h. The reaction mixture is allowed to cool to rt, transferred to a Parr bottle and treated with 10% Pd/C catalyst (1.25 g). The bottle is placed under an atmosphere of hydrogen (53 psi) for 16 h. The mixture is filtered through Celite, and fresh catalyst (10% Pd/C, 1.25 g) is added. Hydrogenolysis continues overnight. The process is repeated three more times until the hydrogenolysis is complete. The final mixture is filtered through Celite and concentrated in vacuo. The residue is purified by flash chromatography on silica gel. Elution with CHCl₃—MeOH—NH₄OH (90:9.5:0.5) affords Int 7 as a white solid (46% yield): ¹H NMR (CDCl₃) δ 5.6-5.5, 3.8-3.7, 3.3-3.2, 2.8-2.7, 2.0-1.8, 1.7-1.5, 1.5.

### Step H

### Preparation of exo-3-amino-1-azabicyclo[2.2.1]heptane bis(hydro-para-toluenesulfonate)

Para-toluenesulfonic acid monohydrate (1.46 g, 7.68 mmol) is added to a stirred solution of Int 7 (770 mg, 3.63 mmol) in EtOH (50 mL). The reaction mixture is heated to reflux for 10 h, followed by cooling to rt. The precipitate is collected by vacuum filtration and washed with cold EtOH to give exo-[2.2.1]-Amine as a white solid (84% yield): ¹H NMR (CD₃OD) δ 7.7, 7.3, 3.9-3.7, 3.7-3.3, 3.2, 2.4, 2.3-2.2, 1.9-1.8.

### Synthesis of endo-3-amino-1-azabicyclo[2.2.1]heptane as the bis(hydro para-toluenesulfonate) salt (endo-[2.2.1]-Amine)

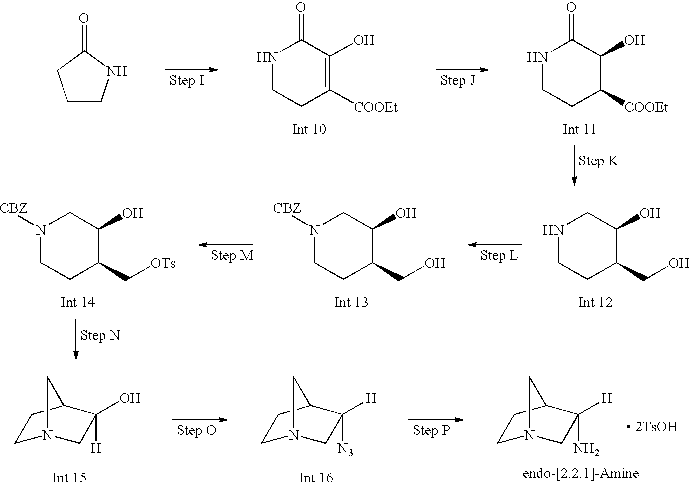

### Step I

### Preparation of ethyl 5-hydroxy-6-oxo-1,2,3,6-tetrahydropyridine-4-carboxylate (Int 10)

Absolute EtOH (92.0 mL, 1.58 mol) is added to a mechanically stirred suspension of potassium ethoxide (33.2 g, 395 mmol) in dry toluene (0.470 L). When the mixture is homogeneous, 2-pyrrolidinone (33.6 g, 395 mmol) is added, and then a solution of diethyl oxalate (53.1 mL, 390 mmol) in toluene (98 mL) is added via an addition funnel. After complete addition, toluene (118 mL) and EtOH (78 mL) are added sequentially. The mixture is heated to reflux for 18 h. The mixture is cooled to rt and aqueous HCl (150 mL of a 6.0 M solution) is added. The mixture is mechanically stirred for 15 min. The aqueous layer is extracted with CH₂Cl₂, and the combined organic layers are dried (MgSO₄), filtered and concentrated in vacuo to a yellow residue. The residue is recrystallized from EtOAc to afford Int 10 as a yellow solid (38% yield): ¹H NMR (CDCl₃) δ 11.4, 7.4, 4.3, 3.4, 2.6, 1.3.

### Step J

### Preparation of ethyl cis-3-hydroxy-2-oxopiperidine-4-carboxylate (Int 11)

A mixture of Int 10 (15 g, 81 mmol) and 5% rhodium on carbon (2.0 g) in glacial acetic acid is placed under an atmosphere of hydrogen (52 psi). The mixture is shaken for 72 h. The mixture is filtered through Celite, and the filtrate is concentrated in vacuo to afford Int 11 as a white solid (98% yield): ¹H NMR (CDCl₃) δ 6.3, 4.2, 4.0-3.8, 3.4, 3.3-3.2, 2.2, 1.3.

### Step K

### Preparation of cis-4-(hydroxymethyl)piperidin-3-ol (Int 12)

Int 11 (3.7 g, 19.9 mmol) as a solid is added in small portions to a stirred solution of LiAlH₄ in THF (80 mL of a 1.0 M solution) in an ice-water bath. The mixture is warmed to rt, and then the reaction is heated to reflux for 48 h. The mixture is cooled in an ice-water bath before water (3.0 mL, 170 mmol) is added dropwise, followed by the sequential addition of NaOH (3.0 mL of a 15% (w/v) solution) and water (9.0 mL, 500 mmol). Excess K₂CO₃ is added, and the mixture is stirred vigorously for 15 min. The mixture is filtered, and the filtrate is concentrated in vacuo to afford Int 12 as a yellow powder (70% yield): ¹H NMR (DMSO-d₆) δ 4.3, 4.1, 3.7, 3.5-3.2, 2.9-2.7, 2.5-2.3, 1.5, 1.3.

### Step L

### Preparation of benzyl cis-3-hydroxy-4-(hydroxymethyl)piperidine-1-carboxylate (Int 13)

N-(benzyloxy carbonyloxy)succinimide (3.04 g, 12.2 mmol) is added to a stirred solution of Int 12 (1.6 g, 12.2 mmol) in saturated aqueous NaHCO₃ (15 mL) at rt. The mixture is stirred at rt for 18 h. The organic and aqueous layers are separated. The aqueous layer is extracted with ether (3×). The combined organic layers are dried over anhydrous K₂CO₃, filtered and concentrated in vacuo to afford Int 13 as a yellow oil (99% yield): ¹H NMR (CDCl₃) δ 7.4-7.3, 5.2, 4.3, 4.1, 3.8-3.7, 3.0-2.8, 2.1, 1.9-1.7, 1.4.

### Step M

### Preparation of benzyl cis-3-hydroxy-4-[(4-methylphenyl)sulfonyl oxymethyl]piperidine-1-carboxylate (Int 14)

Para-toluenesulfonyl chloride (1.0 g, 5.3 mmol) is added to a stirred solution of Int 13 (3.6 g, 5.3 mmol) in pyridine (10 mL) in a −15° C. bath. The mixture is stirred for 4 h, followed by addition of HCl (4.5 mL of a 6.0 M solution). CH₂Cl₂ (5 mL) is added. The organic and aqueous layers are separated. The aqueous layer is extracted with CH₂Cl₂. The combined organic layers are washed with brine, dried (MgSO₄), filtered and concentrated in vacuo to afford Int 14 as a colorless oil (78% yield): ¹H NMR (CDCl₃) δ 7.8, 7.4-7.2, 5.1, 4.3-4.2, 4.1, 3.9-3.8, 2.9-2.7, 2.4, 1.9, 1.6-1.3.

### Step N

### Preparation of exo-1-azabicyclo[2.2.1]heptan-3-ol (Int 15)

A mixture of Int 14 (3.6 g, 8.6 mmol) and 10% Pd/C catalyst (500 mg) in EtOH (50 mL) is placed under an atmosphere of hydrogen. The mixture is shaken for 16 h. The mixture is filtered through Celite. Solid NaHCO₃ (1.1 g, 13 mmol) is added to the filtrate, and the mixture is heated in an oil bath at 50° C. for 5 h. The solvent is removed in vacuo. The residue is dissolved in saturated aqueous K₂CO₃ solution. Continuous extraction of the aqueous layer using a liquid-liquid extraction apparatus (18 h), followed by drying the organic layer over anhydrous K₂CO₃ and removal of the solvent in vacuo affords Int 15 as a white solid (91% yield): ¹H NMR δ 3.8, 3.0-2.8, 2.6-2.5, 2.4-2.3, 1.7, 1.1.

### Step O

### Preparation of endo-3-azido-1-azabicyclo[2.2.1]heptane (Int 16)

To a mixture of Int 15 (1.0 g, 8.9 mmol) and triphenyl phosphine (3.0 g, 11.5 mmol) in toluene-THF (50 mL, 3:2) in an ice-water bath are added sequentially a solution of hydrazoic acid in toluene (15 mL of ca. 2 M solution) and a solution of diethyl azadicarboxylate (1.8 mL, 11.5 mmol) in toluene (20 mL). The mixture is allowed to warm to rt and stir for 18 h. The mixture is extracted with aqueous 1.0M HCl solution. The aqueous layer is extracted with EtOAc, and the combined organic layers are discarded. The pH of the aqueous layer is adjusted to 9 with 50% aqueous NaOH solution. The aqueous layer is extracted with CH₂Cl₂ (3×), and the combined organic layers are washed with brine, dried over Na₂SO₄, filtered and concentrated in vacuo. The crude product is purified by flash chromatography on silica gel. Elution with CHCl₃—MeOH—NH₄OH (92:7:1) affords Int 16 as a colorless oil (41% yield): ¹H NMR (CDCl₃) δ 4.1, 3.2, 2.8, 2.7-2.5, 2.2, 1.9, 1.5.

### Step P

### Preparation of endo-3-amino-1-azabicyclo[2.2.1]heptane bis(hydro-para-toluenesulfonate)

A mixture of Int 16 (250 mg, 1.8 mmol) and 10% Pd/C catalyst (12 mg) in EtOH (10 mL) is placed under an atmosphere of hydrogen (15 psi). The mixture is stirred for 1 h at rt. The mixture is filtered through Celite, and the filtrate is concentrated in vacuo. The residue is dissolved in EtOH (10 mL) and para-toluenesulfonic acid monohydrate (690 mg, 3.7 mmol) is added. The mixture is stirred for 30 min, and the precipitate is filtered. The precipitate is washed sequentially with cold EtOH and ether. The precipitate is dried in vacuo to afford endo-[2.2.1]-Amine as a white solid (85% yield): ¹H NMR (CD₃OD) δ 7.7, 7.3, 4.2, 3.9, 3.6-3.4, 3.3-3.2, 2.4, 2.3, 2.1.

### Preparation of tert-butyl (1S, 2R, 4R)-2-amino-7-azabicyclo[2.2.1]heptane-7-carboxylate

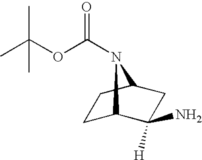

Methyl propiolate (52 ml, 0.583 mol) is combined with recrystallized N-bromo-succinimide (120 g, 0.674 mol) in 1,700 ml acetone under nitrogen. The solution is treated with silver nitrate (9.9 g, 0.0583 mol) neat in a single lot and the reaction is stirred 6 h at RT. The acetone is removed under reduced pressure (25° C., bath temperature) to provide a gray slurry. The slurry is washed with 2×200 ml hexane, the gray solid is removed by filtration, and the filtrate is concentrated in vacuo to provide 95 g of a pale yellow oily residue. The crude material is distilled via short path under reduced pressure (65° C., about 25 mm Hg) into a dry ice/acetone cooled receiver to give 83.7 g (88%) of methyl-3-bromo-propiolate as a pale yellow oil. Anal. calc'd for C₄H₃BrO₂: C, 29.48; H, 1.86. Found: C, 29.09; H, 1.97.

Methyl-3-bromo-propiolate (83.7 g, 0.513 mol) is added to N-t-butyloxy-pyrrole (430 ml, 2.57 mol) under nitrogen. The dark mixture is warmed in a 90° C. bath for 30 h, is cooled, and the bulk of the excess N-t-butyloxy-pyrrole is removed in vacuo using a dry ice/acetone condenser. The dark oily residue is chromatographed over 1 kg silica gel (230-400 mesh) eluting with 0-15% EtOAc/hexane. The appropriate fractions are combined and concentrated to afford 97 g (57%) of 7-tert-butyl 2-methyl 3-bromo-7-azabicyclo[2.2.1]hepta-2,5-diene-2,7-dicarboxylate as a dark yellow oil. HRMS (FAB) calc'd for C₁₃H₁₆BrNO₄+H: 330.0341, found 330.0335 (M+H)⁺.

7-tert-Butyl 2-methyl 3-bromo-7-azabicyclo[2.2.1]hepta-2,5-diene-2,7-dicarboxylate (97 g, 0.294 mol) is added to 10% Pd/C (6.8 g) in 900 ml absolute EtOH in a PARR bottle. The suspension is diluted with a solution of NaHCO₃ (25 g, 0.301 mol) in 250 ml water and the mixture is hydrogenated at 50 PSI for 2.5 h. The catalyst is removed by filtration, is washed with fresh EtOH, and the filtrate is concentrated in vacuo to give a residue. The residue is partitioned between 1×200 ml saturated NaHCO₃ and CH₂Cl₂ (4×100 ml). The combined organic layer is dried over 1:1 anhydrous K₂CO₃/anhydrous MgSO₄ and concentrated in vacuo to afford 72.8 g (98%) of (+/−) endo-7-tert-butyl 2-methyl 7-azabicyclo[2.2.1]heptane-2,7-dicarboxylate. MS (EI) for C₁₄H₂₂O₄, m/z: 255 (M)⁺.

(+/−)Endo-7-tert-butyl 2-methyl 7-azabicyclo[2.2.1]heptane-2,7-dicarboxylate (72.8 g, 0.285 mol) is dissolved in 1000 ml dry MeOH in a dried flask under nitrogen. The solution is treated with solid NaOMe (38.5 g, 0.713 mol) neat, in a single lot and the reaction is warmed to reflux for 4h. The mixture is cooled to 0° C., is treated with 400 ml water, and the reaction is stirred 1 h as it warms to RT. The mixture is concentrated in vacuo to about 400 ml and the pH of the aqueous residue is adjusted to 4.5 with 12N HCl. The precipitate is collected and dried. The tan, slightly tacky solid is washed with 2×100 ml 60% ether in hexane and is dried to provide 47 g (68%) of (+/−) exo-7-(tert-butoxycarbonyl)-7-azabicyclo[2.2.1]heptane-2-carboxylic acid as an off-white powder. HRMS (FAB) calc'd for C₁₂H₁₉NO₄+H: 242.1392, found 242.1390 (M+H)⁺.

(+/−)Exo-7-(tert-butoxycarbonyl)-7-azabicyclo[2.2.1]heptane-2-carboxylic acid (103.9 g, 0.430 mol) is combined with TEA (60 ml, 0.430 mol) in 1200 ml dry toluene in a dry flask under nitrogen. The solution is treated drop-wise with diphenylphosphoryl azide (92.8 ml, 0.430 mol), and is allowed to stir for 20 min at RT. The mixture is treated with benzyl alcohol (47.9 ml, 0.463 mol), and the reaction is stirred overnight at 55° C. The mixture is cooled, is extracted successively with 2×500 ml 5% citric acid, 2×500 ml water, 2×500 ml saturated sodium bicarbonate, and 500 ml saturated NaCl. The organic layer is dried over anhydrous MgSO₄ and concentrated in vacuo to an amber oil. The crude material is chromatographed over 900 g silica gel (230-400 mesh), eluting with 10-30% EtOAc/hexane. The appropriate fractions are combined and concentrated to give 106 g (71%) of (+/−) exo-tert-butyl 2-{[(benzyloxy)carbonyl]amino}-7-azabicyclo[2.2.1]heptane-7-carboxylate as a pale oil. ¹H NMR (CDCl₃) δ 1.29-1.60, 1.44, 1.62-2.01, 3.76-3.88, 4.10, 4.24, 5.10, 7.36 ppm.

(+/−) Exo-tert-Butyl 2-{[(benzyloxy)carbonyl]amino}-7-azabicyclo[2.2.1]heptane-7-carboxylate (1.5 g, 4.33 mmol) is combined with 10% Pd/C (150 mg) in 40 ml EtOH in a 250 ml Parr shaker bottle. The mixture is hydrogenated at 50 PSI for 1.5 h. The catalyst is removed by filtration and the filtrate is concentrated in vacuo. The crude material is chromatographed over 30 g silica gel (230-400 mesh), eluting with 7% MeOH/CH₂Cl₂+1% conc. NH₄OH. The appropriate fractions are combined and concentrated to provide 606 mg (66%) of (+/−) exo-tert-butyl 2-amino-7-azabicyclo[2.2.1]heptane-7-carboxylate. HRMS (FAB) calcd for C₁₁H₂₀N₂O₂+H: 213.1603, found 213.1580 (M+H)⁺. This racemic mixture will be referenced as (+/−)-7-aza-[2.2.1]-Amine.

Resolution of racemic carboxylate mixture:

The isolated (+/−) exo-tert-butyl 2-{[(benzyloxy)carbonyl]amino}-7-azabicyclo[2.2.1]heptane-7-carboxylate is resolved via preparative chiral HPLC (50×500 mm Chiralcel OJ column, 30 deg. C., 70 mL/min. 10/90 (v/v) isopropanol/heptane). The resolution affords 40 g of tert-butyl (1S, 2R, 4R)-(+)-2{[(benzyloxy)carbonyl]amino}-7-azabicyclo[2.2.1]heptane-7-carboxylate and 42 g of tert-butyl-(1R, 2S, 4S)(−)-2{[(benzyloxy)carbonyl]amino}-7-azabicyclo[2.2.1]heptane-7-carboxylate.

The 2R enantiomer is triturated with 40 ml ether followed by 40 ml hexane (to remove lingering diastereo and enantiomeric impurities) and is dried to afford 30 g (56%) of purified tert-butyl (1S, 2R, 4R)-(+)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=22, (c 0.42, chloroform).

The 2S enantiomer is triturated with 40 ml ether followed by 40 ml hexane to give 35 g (66%) of purified tert-butyl (1R, 2S, 4S)-(−)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=−23, (c 0.39, chloroform).

Preparation of (2R)-7-aza-[2.2.1]-Amine.

tert-Butyl (1S, 2R, 4R)-(+)-2{[(benzyloxy)carbonyl]amino}-7-azabicyclo[2.2.1]heptane-7-carboxylate (9.5 g, 27.4 mmol) is combined with 950 mg 10% Pd/C in 75 ml absolute EtOH in a 500 ml Parr bottle. The reaction mixture is hydrogenated at 50 PSI for 3 h, the catalyst is removed by filtration, and the filter cake is washed with MeOH. The filtrate is concentrated in vacuo to give 6.4 g of a residue. The crude material is chromatographed over 200 g silica gel (230-400 mesh) eluting with 7% CH₃OH/CHCl₃ containing 1% conc. NH₄OH. The appropriate fractions are combined and concentrated to give 5.61 g (96%) of tert-butyl-(1S, 2R, 4R)-(+)-2-amino-7-azabicyclo[2.2.1]heptane-7-carboxylate as a pale oil. MS (EI) for C₁₁H₂₀N₂O₂, m/z: 212 (M)⁺. [α]²⁵D=9, (c 0.67, CHCl₃). This compound will be referenced as (2R)-7-aza-[2.2.1]-Amine.

### Preparation of 1-azabicyclo[3.2.1]octan-3-amine

The exo- and endo-1-azabicyclo[3.2.1]octan-3-amines are prepared from 1-azabicyclic[3.2.1]octan-3-one (Thill, B. P., Aaron, H. S., J. Org. Chem., 4376-4380 (1968)) according to the general procedure as discussed in Lewin, A. H., et al., J. Med. Chem., 988-995 (1998).

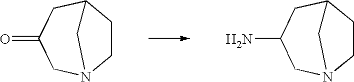

### exo-1-Azabicyclo[3.2.1]octan-3-amine dihydrochloride (exo-[3.2.1]-Amine)

A mixture of 1-azabicyclo[3.2.1]octan-3-one hydrochloride (2.80 g, 17.3 mmol), ethanol (25 mL), and hydroxylamine hydrochloride (1.56 g, 22.4 mmol) is treated with sodium acetate trihydrate (7.07 g, 51.2 mmol). The mixture is stirred for 3 h and evaporated in vacuo. The residue is diluted with CH₂Cl₂, treated with charcoal, filtered and evaporated. The resulting material is taken up in 1-propanol (45 mL) and heated in a 100° C. oil bath. The solution is treated with sodium metal (6.4 g in portions). Heating is continued for 3 h and the mixture cooled to rt. Water is added carefully and the organic layer is extracted, dried (MgSO₄), filtered, acidified with MeOH/HCl(g), and evaporated. 2-Propanol is added and the resulting solid is filtered and dried in vacuo to give exo-[3.2.1]-Amine in 49% yield. MS for C₇H₁₄N₂.(HCl)₂ (ESI) (M+H)⁺ m/z=127.

### endo-1-Azabicyclo [3.2.1]octan-3-amine dihydrochloride (endo-[3.2.1]-Amine)

A mixture of 1-azabicyclo[3.2.1]octan-3-one hydrochloride (2.80 g, 17.3 mmol), ethanol (25 mL), and hydroxylamine hydrochloride (1.56 g, 22.4 mmol) is treated with sodium acetate trihydrate (7.07 g, 51.2 mmol). The mixture is stirred for 3 h and evaporated in vacuo. The residue is diluted with CH₂Cl₂, treated with charcoal, filtered and evaporated. The resulting oxime (3.1 mmol) is treated with acetic acid (30 mL) and hydrogenated at 50 psi over PtO₂ (50 mg) for 12 h. The mixture is then filtered and evaporated. The residue is taken up in a minimal amount of water (6 mL) and the pH is adjusted to >12 using solid NaOH. The mixture is then extracted with ethyl acetate (4×25 mL), dried (MgSO₄), filtered, treated with ethereal HCl, and evaporated to give endo-[3.2.1]-Amine.

### Preparation of the 3R,5R-[3.2.1]-Amine

This amine can also be prepared according to the following method:

(3S)-1-[(S)-1-Phenethyl]-5-oxo-3-pyrrolidine-carboxylic acid:

According to the literature procedure (Nielsen et al. J. Med. Chem 1990, 70-77), a mixture of itaconic acid (123.2 g, 946.7 mmol) and (S)-(−)-α-methyl benzylamine (122 mL, 946 mmol) are heated (neat) in a 160° C. oil bath for 4 h. Upon cooling, MeOH (˜200 mL) is added and the resulting solid collected by filtration. The solid is treated with EtOH (˜700 mL) and warmed using a steam bath until ˜450 mL solvent remained. After cooling to rt, the solid product is collected and dried to afford 83.2 g as a crystalline solid: [α]²⁵_{D}=−80 (c 0.97, DMSO). ¹H NMR (400 MHz, DMSO-d₆) δ 12.66, 7.20-7.40, 5.23, 3.40-3.55, 3.10-3.25, 2.40-2.65, 1.45; MS (EI) m/z 233 (M⁺).

(3S)-1-[(S)-1-Phenethyl]-3-(hydroxymethyl)pyrrolidine:

A suspension (3S)-1-[(S)-1-phenethyl]-5-oxo-3-pyrrolidine-carboxylic acid (82.3 g, 352.3 mmol) in Et₂O (200 mL) is added in small portions to a slurry of LiAlH₄ (17.4 g, 459 mmol) in Et₂O (700 mL). The mixture begins to reflux during the addition; the addition funnel containing the suspension is rinsed with Et₂O (2×50 mL). The mixture is heated in a 50° C. oil bath for an additional 2 h, allowed to cool to rt, and further cooled using an ice bath. The mixture is carefully treated with H₂O (62 mL). The resulting precipitate is filtered, rinsed with Et₂O, and discarded. The filtrate is concentrated to an oil. When EtOAc is added to the oil, a solid began to form. Hexane is added, and the mixture is filtered and the solid is dried to afford 43.3 g of the desired product. [α]²⁵_{D}=−71 (c 0.94, CHCl₃); ¹H NMR (400 MHz, CDCl₃) δ 7.20-7.45, 3.60-3.70, 3.40-3.60, 3.19, 3.05-3.15, 2.35-2.55, 2.25-2.35, 1.95-2.10, 1.75-1.90, 1.42; HRMS (FAB) calcd for C₁₃H₁₉NO (MH⁺) 206.1545, found 206.1532.

(3R)-1-[(S)-1-Phenethyl]-3-(cyanomethyl)pyrrolidine:

A solution of (3S)-1-[(S)-1-phenethyl]-3-(hydroxymethyl)pyrrolidine (42.75 g, 208.2 mmol) in chloroform (350 mL) is heated to reflux under N₂. The solution is treated with a solution of thionyl chloride (41.8 mL, 573 mmol) in chloroform (40 mL) dropwise over 45 min. The mixture is stirred for an additional 30 min, is cooled and concentrated. The residue is diluted with H₂O (˜200 mL), 1 N NaOH is added until the pH ˜8 (pH paper). A small portion (˜50 mL) of sat. NaHCO₃ is added, and the basic mixture is extracted with EtOAc (3×400 mL), washed with brine, dried (MgSO₄), filtered and concentrated to give 46.51 g of (3S)-1-[(S)-1-phenethyl]-3-(chloromethyl)pyrrolidine: MS (ESI+) m/z 224.2 (MH⁺). The chloride (46.4 g, 208 mmol) is transferred to a flask, DMSO (200 mL) is added, and the solution is treated with NaCN (17.84 g, 363.9 mmol). The mixture is heated under N₂ in a 100° C. oil bath overnight and is cooled. The brown mixture is poured into H₂O (300 mL) and is extracted with EtOAc (1000 mL in portions). The combined organic layer is washed with H₂O (6×˜50 mL), brine (˜100 mL), dried (MgSO₄), filtered and concentrated to give 40.61 g of an oil: ¹H NMR (400 MHz, CDCl₃) δ 7.20-7.40, 3.26, 2.70-2.85, 2.40-2.60, 2.27, 2.10-2.20, 1.50-1.70, 1.41; MS (ESI+) for m/z 215.2 (M+H⁺).

(3R)-Methyl 1-[(S)-1-phenylethyl]pyrrolidine-3-acetate:

Acetyl chloride (270 mL, 3.8 mol) is carefully added to a flask containing chilled (0° C.) methanol (1100 mL). After the addition is complete, the acidic solution is stirred for 45 min (0° C.) and then (3R)-1-[(S)-1-phenethyl]-3-(cyanomethyl)pyrrolidine (40.50 g, 189.0 mmol) in methanol (200 mL) is added. The ice bath is removed and the mixture is stirred for 100 h at rt. The resulting suspension is concentrated. Water (˜600 mL) is added, the mixture stirred for 45 min and then the pH is adjusted (made basic) through the addition of ˜700 mL sat. aq. NaHCO₃. The mixture is extracted with EtOAc (3×300 mL). The combined organic layers are washed with brine, dried (MgSO₄), filtered through celite, and concentrated to give 36.9 g as an oil: ¹H NMR (400 MHz, CDCl₃) δ 7.20-7.40, 3.69, 3.30-3.40, 2.85-2.95, 2.40-2.70, 2.00-2.20, 1.10-1.65; MS (ESI+) m/z 248.2 (M+H⁺).

(5R)-1-Azabicyclo[3.2.1]octan-3-one hydrochloride:

A solution of (3R)-methyl 1-[(S)-1-phenylethyl]pyrrolidine-3-acetate (25.7 g, 104.0 mmol) in THF (265 mL) is cooled under N₂ in a CO₂/acetone bath. Next, ICH₂Cl (22.7 mL, 312.0 mmol) is added, and the mixture stirred for 30 min. A solution of 2.0M lithium diisopropylamide (heptane/THF/ethylbenzene, 156 mL, 312 mmol) is added slowly over 30 min. The internal temperature reached a maximum of −40° C. during this addition. After 1 h, sat. NH₄Cl (100 mL) is added and the mixture is allowed to warm to rt. The organic layer is separated, dried (MgSO₄), filtered, and concentrated. The resulting foam is chromatographed (300 g SiO₂, CHCl₃—MeOH—NH₄OH (89:10:1) followed by CHCl₃—MeOH (3:1). The product fractions are pooled and concentrated to afford (5R)-3-oxo-1-[(1S)-1-phenylethyl]-1-azoniabicyclo[3.2.1]octane chloride (10.1 g) as a foam (MS (ESI+) m/z 230.1 (M+H⁺). This foam (10.1 g, 38.0 mmol) is taken up in MeOH (500 mL), 10% Pd(C) (3.0 g) added and the mixture is hydrogenated (45 psi) overnight. The mixture is filtered and re-subjected to the reduction conditions (9.1 g, 10% Pd/C, 50 psi). After 5 h, TLC indicates the consumption of the (5R)-3-oxo-1-[(1S)-1-phenylethyl]-1-azoniabicyclo[3.2.1]octane chloride. The mixture is filtered, concentrated and triturated (minimal iPrOH) to give 3.73 g in two crops, as a solid: [α]²⁵_{D}=33 (c 0.97, DMSO); HRMS (FAB) calcd for C₇H₁₁NO (M+H⁺) 126.0919, found 126.0937.

exo-(3R,5R)-1-azabicyclo[3.2.1]octan-3-amine dihydrochloride:

To a flask containing (5R)-1-azabicyclo[3.2.1]octan-3-one hydrochloride (3.64 g, 22.6 mmol), hydroxylamine hydrochloride (2.04 g, 29.4 mmol), and ethanol (130 mL) is added sodium acetate trihydrate (9.23 g, 67.8 mmol). The mixture stirred for 3 h, filtered, and concentrated. The resulting solid is taken up in n-propanol (100 mL) and sodium (˜13.6 g, 618 mmol) is added in 20-25 portions. The reaction spontaneously begins to reflux, and the reaction is heated in an oil bath (100° C.). The addition is complete in ˜20 min and the mixture solidifies after ˜40 min. The oil bath is removed and n-propanol (2×25 mL) is added dissolving the remaining sodium metal. The mixture is carefully quenched through the dropwise addition of H₂O (100 mL). Saturated aq. NaCl (20 mL) is added, and the layers are separated. The organic layer is dried (MgSO₄), filtered, treated with freshly prepared MeOH/HCl, and concentrated. The resulting solid is triturated with 30 mL EtOH, filtered and dried in vaccuo to afford 3.51 g of the (3R, 5R)-[3.2.1]-Amine as a solid: [α]²⁵_{D}=−3 (c 0.94, DMSO); ¹H NMR (400 MHz, DMSO-d₆) δ 3.60-3.80, 2.95-3.10, 2.65-2.75, 1.90-2.15, 1.70-1.90; HRMS (FAB) calcd for C₇H₁₄N₂ (M+H⁺) 127.1235, found 127.1235.

The following examples are provided as examples and are not intended to limit the scope of this invention to only those provided examples and named compounds. Also, the salts made in the examples are only exemplary and are not intended to limit the invention. Any pharmaceutically acceptable salt can be made by one of ordinary skill in the art. Further, the naming of specific stereoisomers is for exemplification, and is not intended to limit in anyway the scope of the invention. The invention includes the following examples in pure stereoisomeric form or as racemic mixtures.

### EXAMPLE 1

### N-[(3R)-1-azabicyclo[2.2.2]oct-3-yl]thieno[3,2-c]pyridine-6-carboxamide dihydrochloride

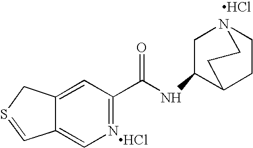

Glyoxylic acid monohydrate (20.3 g, 221 mmol) and benzyl carbamate (30.6 g, 202 mmol) are added to ether (200 mL). The solution is allowed to stir for 24 h at rt. The resulting thick precipitate is filtered, and the residue is washed with ether, affording ([(benzyloxy)carbonyl]amino)(hydroxy)acetic acid (C150) as a white solid (47% yield). MS (CI) for C₁₀H₁₁NO₅+H m/z: 226 (M+H)⁺.

C150 (11.6 g, 51.5 mmol) is dissolved in absolute MeOH (120 mL) and chilled in an ice bath. Concentrated sulfuric acid (2.0 mL) is carefully added drop-wise. The ice bath is allowed to expire as the solution stirred for 2 days. The reaction is quenched by pouring onto a mixture of 500 g ice with saturated NaHCO₃ solution (400 mL). The solution is extracted with EtOAc (3×300 mL), and the combined organic layer is dried (MgSO₄), filtered, and concentrated to a pale oil that crystallized upon standing, giving methyl([(benzyloxy)carbonyl]amino)(methoxy)acetate (C151) as a white solid (94% yield). Analysis calculated for C₁₂H₁₅NO₅: C, 56.91; H, 5.97; N, 5.53, found: C, 56.99; H, 6.02; N, 5.60.

C151 (11.76 g, 46.4 mmol) is dissolved in toluene (50 mL) under N₂ and heated to 70° C. Phosphorous trichloride (23.2 mL, 46.4 mmol) is added drop-wise via syringe, and the solution is stirred for 18 h at 70° C. Trimethyl phosphite (5.47 mL, 46.4 mmol) is then added drop-wise, and stirring continued for an additional 2 h at 70° C. The mixture is concentrated in vacuo to an oil, and the crude material is dissolved in EtOAc (100 mL) and washed with saturated NaHCO₃ (3×50 mL). The organic layer is dried over Na₂SO₄, filtered, and concentrated to a volume of 30 mL. This remaining solution is stirred vigorously while hexane is added until a precipitate formed. The precipitated solid is removed by filtration, affording methyl ([(benzyloxy)carbonyl]amino) (dimethoxyphosphoryl)acetate (C152) as a white solid (84% yield). MS (EI) for C₁₃H₁₈NO₇P, m/z: 331 (M)⁺.

C152 (12.65 g, 38.2 mmol) and acetic anhydride (9.02 mL, 95.5 mmol) in MeOH (100 mL) are added to a Parr flask. The solution is hydrogenated with 10% Pd/C catalyst (0.640 g) at 45 PSI for 3 h. The catalyst is filtered off, and the filtrate is concentrated in vacuo to an oil. The oil is placed under reduced pressure and solidified as the reduced pressure is applied. The white residue is dissolved in a small amount of EtOAc and stirred vigorously while pentane is added until a precipitate began to form. The precipitate is removed by filtration to give methyl (acetylamino)(dimethoxyphosphoryl)acetate (C153) as a white powder (87% yield). MS (CI) for C₇H₁₄NO₆P, m/z: 240 (M+H)⁺.

2,3-Thiophene dicarboxaldehyde (1.40 g, 9.99 mmol) is dissolved in CH₂Cl₂ (100 mL) and the flask is placed in an ice bath. C153 (2.63 g, 11.0 mmol) is dissolved in CH₂Cl₂ (50 mL), DBU (1.65 mL, 11.0 mmol) is added, and this solution is added drop-wise to the chilled thiophene solution. The reaction mixture is stirred for 1 h while the flask is in an ice bath and then over night at rt. The reaction is concentrated in vacuo, and the crude material is chromatographed over 300 g slurry-packed silica eluting with 50% EtOAc/hexane. The fractions are collected in two different groups to obtain the desired compounds. Each group of fractions is combined and concentrated separately. Methyl thieno[2,3-c]pyridine-5-carboxylate (C154) elutes first and the appropriate fractions are concentrated to give a white solid (41% yield). The second group of appropriate fractions are collected and concentrated to give methyl thieno[3,2-c]pyridine-6-carboxylate (C155) as a yellow solid (38% yield). MS (EI) for C154 for C₉H₇NO₂S, M/z: 193 (M)⁺. MS (EI) for C155 for C₉H₇NO₂S, m/z: 193 (M)⁺.

C155 (736 mg, 3.8 mmol) is dissolved in MeOH (16 mL) with water (2 mL). 2M NaOH (2.0 mL, 4.0 mmol) is added drop-wise and the solution stirred at rt. After 2 days (complete disappearance of ester by TLC), the reaction is concentrated in vacuo. The residue is dissolved in water (12 mL), and the pH is adjusted to 3.5 with 10% HCl. The precipitated solid is removed by filtration, and the solid is rinsed with ether, affording thieno[3,2-c]pyridine-6-carboxylic acid (C156) as a white solid (58% yield). HRMS (FAB) calculated for C₈H₅NO₂S+H: 180.0119, found 180.0123 (M+H)⁺.

Method A:

Thieno[3,2-c]pyridine-6-carboxylic acid (185 mg, 1.03 mmol) is combined with TEA (0.167 ml, 1.20 mmol) in CH₂Cl₂ (4 ml). Bis(2-oxo-3-oxazolidinyl)phosphinic chloride (308 mg, 1.20 mmol) is added portionwise and the solution is stirred at rt for 30 min. 0.5M free-based (R)-(3)-aminoquinuclidine solution in DMF (3 ml, 1.5 mmol) is added drop-wise and the reaction stirred for 4 h. The reaction mixture is poured through pre-washed Amberjet 4400 OH Strongly Basic Anion Exchanger resin directly into pre-washed AG 50W-X2 Hydrogen Form resin. The acid resin is washed with MeOH (100 ml), and the product eluted with 10% TEA/MeOH solution (100 ml). The solution is concentrated in vacuo to a glass. The crude material is chromatographed over 10 g slurry-packed silica, eluting with 1% NH₄OH/10% MeOH/CH₂Cl₂ into 100 mm fractions. The appropriate fractions arecollected and concentrated in vacuo to yield 0.115 g (39%) of glass. The glass is dissolved in 1M HCl in MeOH (1.6 ml) and stirred for 2 h. IPA (2 ml) and Et₂O (4 ml) are added to enhance precipitation. The precipitate is isolated via filtration and dried to afford 116 mg (31%) of as a white salt. HRMS (FAB) calcd for C₁₅H₁₇N₃OS+H: 288.1170, found 288.1174 (M+H)⁺.

### EXAMPLE 2

### N-[(3S)-1-azabicyclo[2.2.2]oct-3-yl]thieno[3,2-c]pyridine-6-carboxamide dihydrochloride

Example 2 can be prepared using Method A, making non-critical changes and using (S)-3-aminoquinuclidine free base.

### EXAMPLE 3

### N-[(3R)-1-azabicyclo[2.2.2]oct-3-yl]-3-bromo-1-benzofuran-5-carboxamide

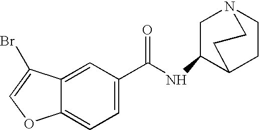

4-Hydroxybenzoic acid (34.5 g, 250 mmol) is suspended in MeOH (500 mL), treated with sodium iodide (34.5 g, 250 mmol) and NaOH (20 g, 500 mmol) and cooled to 0° C. Sodium hypochlorite (Clorox bleach) (423 mL, 250 mmol) is added slowly dropwise at 0-5° C. and the mixture is stirred for 1 h. The mixture is treated with saturated Na₂S₂O₃ (135 mL) and water (135 mL) and stirred overnight as the cooling bath expired. The mixture is acidified to pH 3.5 with concentrated HCl and the resulting precipitate filtered off and discarded. The filtrate is concentrated to dryness, partitioned between water (300 mL) and EtOAc (1×500 mL, then 3×300 mL), dried over anhydrous Na₂SO₄ and concentrated to afford 59.6 g (90%) of essentially pure 4-hydroxy-3-iodobenzoic acid as a white solid. MS (ESI): 262.9 (M−H)⁻.

4-Hydroxy-3-iodobenzoic acid (59.6 g, 226 mmol) is combined with 3 N methanolic HCl (276 mL, 678 mmol) and heated to 65° C. for 24 h, then concentrated to dryness. The residue is diluted with water, neutralized to pH 7 with 3 N NaOH and the resulting solid collected via filtration. The crude material is adsorbed onto silica gel (230-400 mesh) and chromatographed over 1 kg of silica gel eluting with EtOAc/hexane mixtures. All fractions containing product are combined and concentrated to a solid (47.2 g). The material is recrystallized with EtOAc to afford cleaner material (16.6 g). A second recrystallization of the filtrate in EtOAc resulted in a second solid of comparable purity (6.2 g). The remaining solid (24.5 g) is carried on without further purification. Recrystallized total: 22.8 g (36%) as a white solid. HRMS (FAB) calcd for C₈H₇IO₃+H: 278.9520, found 278.9534 (M+H)⁺.

Methyl 4-hydroxy-3-iodobenzoate (5.56 g, 20 mmol) is combined with trimethylsilylacetylene (3.96 mL, 28 mmol), bis(triphenylphosphine)palladium dichloride (414 mg, 0.6 mmol) and cuprous iodide (57 mg, 0.3 mmol) in THF (20 mL)/CHCl₃ (40 mL) in an oven-dried flask, under nitrogen. Triethylamine (8.7 mL, 62.3 mmol) is added and the mixture heated to 50° C. for 4 h. The mixture is diluted with CHCl₃ (60 mL), washed with 5% HCl (2×40 mL), dried over anhydrous MgSO₄ and concentrated to a brown solid. The crude material is adsorbed onto silica gel and chromatographed over 200 g silica gel, eluting with 15%-30% EtOAc/hexane into 50 mL fractions. The appropriate fractions are combined and concentrated to afford 5.0 g (95%) of methyl 4-hydroxy-3-[(trimethylsilyl)ethynyl]benzoate as an orange solid. HRMS (FAB) calcd for C₁₃H₁₆O₃Si+H: 249.0947, found 249.0955 (M+H)⁺.

Methyl 4-hydroxy-3-[(trimethylsilyl)ethynyl]benzoate (11 g, 44.5 mmol) is combined with diisopropylamine (7.1 ml, 50 mmol) and cuprous iodide (423 mg, 2.2 mmol) in 100 ml MeOH in a flask under nitrogen. The reaction is warmed to 60° C. for 6 h, the volatiles are removed in vacuo, and the brown-green residue is chromatographed over 500 g silica gel (230-400 mesh) eluting with 20% EtOAc/hexane. The appropriate fractions are combined and concentrated to give 2.63 g (34%) of methyl benzofuran-5-carboxylate. ¹H NMR (300 MHz, CDCl₃) δ 3.96, 6.86, 7.55, 7.70, 8.04, 8.36 ppm.

Methyl benzofuran-5-carboxylate (667 mg, 3.8 mmol) is dissolved in 20 ml CH₂Cl₂ in a flask under nitrogen. The solution is treated with bromine (1.2 ml, 22.8 mmol), is layered with 20 ml saturated sodium bicarbonate, and the reaction is stirred gently for 2 h at rt. The reaction is stirred vigorously for 30 min, the layers are separated, and the organic layer is concentrated in vacuo to an amber oil. The residue is dissolved in 30 ml EtOH, the solution is treated with anhydrous K₂CO₃ (3.15 g, 22.8 mmol), and the reaction is stirred vigorously overnight. The insoluble material is removed by filtration, the filtrate is diluted with 3 ml 3N NaOH, and the mixture is stirred 3 h at rt. The mixture is concentrated in vacuo, the residue is dissolved in 10 ml water, and the pH of the solution is adjusted to 2 with 10% aqueous HCl. The precipitate is collected, washed with water, and is dried to afford 880 mg (96%) of 3-bromobenzofuran-5-carboxylic acid as an off-white solid. HRMS (FAB) calcd for C₉H₅BrO₃+H: 240.9501, found 240.9505 (M+H)⁺.

Method B:

3-Bromobenzofuran-5-carboxylic acid (1.0 g, 4.1 mmol) is combined with 3(R)-aminoquinulcidine dihydrochloride (908 mg, 4.6 mmol) and DIEA (2.9 ml, 16.6 mmol) in 10 ml DMF in a dry flask under nitrogen. The mixture is treated with HATU (1.73 g, 4.6 mmol), and the reaction is stirred overnight at rt. The volatiles are removed in vacuo, the residue is partitioned between 50 ml CHCl₃ and 50 ml 1:1 conc. NH₄OH/sat'd NaCl, and the aqueous layer is extracted with 50 ml CHCl₃. The combined organic layer is dried over anhydrous K₂CO₃, is concentrated to dryness, and the residue is chromatographed over 30 g silica gel (230-400 mesh) eluting with 8% MeOH/CHCl₃+0.5% conc. NH₄OH. The appropriate fractions are combined and concentrated to afford 1.34 g (93%) of Example 3 as an off-white solid. HRMS (FAB) calcd for C₁₆H₁₇BrN₂O₂+H: 349.0552, found 349.0555 (M+H)⁺.

### EXAMPLE 4

### N-[(3S)-1-azabicyclo[2.2.2]oct-3-yl]-3-bromo-1-benzofuran-5-carboxamide

Example 4 can be prepared using Method B, making non-critical changes and using (S)-3-aminoquinuclidine free base.

### EXAMPLE 5

### N-[(3R)-1-azabicyclo[2.2.2]oct-3-yl]-1H-pyrrolo[2,3-c]pyridine-5-carboxamide dihydrochloride

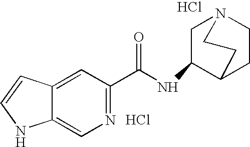

2,4-Lutidine (51.4 mL, 0.445 mole) is added drop-wise to 250 mL fuming sulfuric acid in a flask under N₂ in an ice bath. The solution is treated portionwise with potassium nitrate (89.9 g, 0.889 mole) over a 15 min period. The reaction is stirred 1 h in an ice bath, 2 h at rt, is gradually warmed in a 100° C. oil bath for 5 h, and then in a 130° C. oil bath for 4 h. The mixture is cooled, is poured into 1000 mL ice, and the mixture is neutralized with NaHCO₃ (1,100 g, 13.1 mole). The precipitated Na₂SO₄ is removed by filtration, the solid is washed with 500 mL water and the filtrate is extracted with 4×500 mL ether. The combined organic layer is dried over anhydrous MgSO₄ and is concentrated in vacuo to a yellow oil (50 g). The crude oil is distilled under vacuum to provide three fractions: 16 g recovered 2,4-lutidine (85° C.), 16 g 2,4-dimethyl-3-nitro-pyridine (C169) contaminated with 25% 2,4-dimethyl-5-nitro-pyridine (135-145° C.), and 16 g 2,4-dimethyl-5-nitro-pyridine (C170) contaminated with 2,4-dimethyl-3-nitropyridine (145-153° C.). ¹H NMR of C169 (CDCl₃) δ 2.33 (s, 3 H), 2.54 (s, 3 H), 7.10 (d, J=5 Hz, 1 H), 8.43 (d, J=5 Hz, 1 H) ppm. ¹H NMR of C170 (CDCl₃) δ 2.61 (s, 3 H), 2.62 (s, 3 H), 7.16 (s, 1 H), 9.05 (s, 1 H) ppm.

C170/C169 (75:25) (5.64 g, 37 mmol) is combined with benzeneselenic anhydride (8.2 g, 22.8 mmol) in 300 mL dioxane in a flask under N₂. The reaction is warmed to reflux for 10 h, is cooled, and is concentrated to a dark yellow oil. The oil is chromatographed over 250 g silica gel (230-400 mesh) eluting with 15% EtOAc/hexane. The appropriate fractions are concentrated to afford 2-formyl-4-methyl-5-nitropyridine (C171) (66% yield). HRMS (EI) calculated for C₇H₆N₂O₃: 166.0378, found 166.0383 (M⁺).

C171 (1.15 g, 6.9 mmol), p-toluene sulfonic acid (41 mg, 0.22 mmol), and ethylene glycol (1.41 mL, 25 mmol) are added to 25 mL toluene in a flask equipped with a Dean-Starke trap. The reaction is warmed to reflux for 2 h, is cooled to rt, and is concentrated in vacuo to an oily residue. The crude oil is chromatographed over 40 g silica gel (Biotage), eluting with 20% EtOAc/hexane. The appropriate fractions are combined and concentrated to afford 2-(1,3-dioxolan-2-yl)-4-methyl-5-nitropyridine (C172) (90% yield). MS (EI) for C₉H₁₀N₂O₄, m/z: 210 (M)⁺.

C172 (1.3 g, 6.2 mmol) and DMF dimethyl acetal (1.12 mL, 8.4 mmol) are added to 15 mL DMF under N₂. The reaction is warmed to 90° C. for 3 h, is cooled, and the reaction is concentrated in vacuo. The residue is combined with 1.25 g 5% Pd/BaSO₄ in 20 mL EtOH in a 250 mL Parr shaker bottle and the mixture is hydrogenated at ambient pressure until uptake ceased. The catalyst is removed by filtration, and the filtrate is combined with 500 mg 10% Pd/C catalyst in a 250 mL Parr shaker bottle. The mixture is hydrogenated at ambient pressure for 1 h. No additional hydrogen uptake is observed. The catalyst is removed by filtration, and the filtrate is concentrated in vacuo to a tan solid. The crude material is chromatographed over 50 g silica gel (230-400 mesh), eluting with 7% MeOH/CH₂Cl₂. The appropriate fractions are combined and concentrated to afford 5-(1,3-dioxolan-2-yl)-1H-pyrrolo[2,3-c]pyridine (C173) (69% yield). MS for C₁₀H₁₀N₂O₂, (EI) m/z: 190 (M)⁺.

C173 (800 mg, 4.21 mmol) is dissolved in 44 mL 10% aqueous acetonitrile. p-Toluene sulfonic acid (630 mg, 3.3 mmol) is added, and the mixture is heated to reflux for 5 h. The mixture is cooled to rt, is concentrated in vacuo, and the resultant residue is diluted with 15 mL saturated NaHCO₃. A pale yellow solid is collected, washed with water, and is dried to afford 1H-pyrrolo[2,3-c]pyridine-5-carbaldehyde (C174) (81% yield). HRMS (FAB) calculated for C₈H₆N₂O+H: 147.0558, found 147.0564 (M+H)⁺.

C174 (500 mg, 3.42 mmol) is dissolved in 1.5 mL formic acid. The solution is cooled to in an ice bath, 30% aqueous hydrogen peroxide (722 μL, 6.8 mmol) is added drop-wise, and the reaction is stirred 1 h in an ice bath, and allowed to stand overnight at 5° C. The mixture is diluted with water, the solid is collected, washed with water and is dried to give 522 mg of an off-white solid. The formate salt is added to 7 mL water, 3 mL 2N NaOH is added, and the pH is adjusted to 3 with 5% aqueous HCl. The precipitate is collected and is dried to afford 1H-pyrrolo[2,3-c]pyridine-5-carboxylic acid (C176) (67% yield). HRMS (FAB) calculated for C₈H₆N₂O₂+H: 163.0508, found 163.0507 (M+H)⁺.

Example 5 is obtained as a white solid (40% yield) using acid C176 using Method B with non-critical changes. HRMS (FAB) calculated for C₁₅H₁₈N₄O+H: 271.1559, found 271.1562 (M+H)⁺.

### EXAMPLE 6

### N-[(3S)-1-azabicyclo[2.2.2]oct-3-yl]-1H-pyrrolo[2,3-c]pyridine-5-carboxamide dihydrochloride

Example 6 can be prepared using Method B, making non-critical changes and using (S)-3-aminoquinuclidine free base.

### EXAMPLE 7

### N-[(3R)-1-azabicyclo[2.2.2]oct-3-yl]-1-methyl-1H-pyrrolo[2,3-c]pyridine-5-carboxamide dihydrochloride

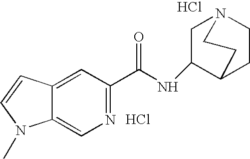

C173 (1.05 g, 5.52 mmol) is dissolved in 20 mL THF in a dried flask under N₂. 60% Sodium hydride (243 mg, 6.07 mmol) is added, the reaction is stirred 30 min, methyl iodide (360 μL, 5.8 mmol) is added, and the reaction is stirred overnight at rt. The reaction is concentrated in vacuo and the residue is partitioned between 10 mL saturated NaCl and CH₂Cl₂ (4×10 mL). The combined organic layer is dried over anhydrous K₂CO₃ and is concentrated in vacuo to a tan paste. The crude material is chromatographed over 50 g silica gel (230-400 mesh) eluting with 5% MeOH/CH₂Cl₂. The appropriate fractions are combined and concentrated to afford 5-(1,3-dioxolan-2-yl)-1-methyl-1H-pyrrolo[2,3-c]pyridine (C175) (86% yield). HRMS (FAB) calculated for C₁₁H₁₂N₂O₂+H: 205.0977, found 205.0983.

C175 (920 mg, 4.5 mmol) is dissolved in 25 mL 10% aqueous acetonitrile in a flask. p-Toluene sulfonic acid (630 mg, 3.3 mmol) is added, and the mixture is heated to 90° C. for 8 h. The mixture is cooled to rt, concentrated in vacuo, and the residue is partitioned between 15 mL saturated NaHCO₃ and CH₂Cl₂ (4×10 mL). The combined organic layer is dried over anhydrous K₂CO₃ and is concentrated in vacuo to afford 1-methyl-pyrrolo[2,3-c]pyridine-5-carbaldehyde (C177) (99% yield). HRMS (FAB) calculated for C₉H₈N₂O+H: 161.0715, found 161.0711.

C177 (690 mg, 4.3 mmol) is dissolved in 2 mL formic acid. The solution is cooled in an ice bath, 30% aqueous hydrogen peroxide (970 μmL, 8.6 mmol) is added drop-wise, and the reaction is stirred 1 h in an ice bath, and allow to stand overnight at 5° C. The mixture is concentrated to dryness, is suspended in water, and the pH is adjusted to 7 with 2N NaOH. The mixture is concentrated to dryness, is dissolved in MeOH, and is passed over 15 mL 50W-X2 ion exchange resin (hydrogen form) eluting with 200 mL MeOH followed by 200 mL 5% Et₃N/MeOH. The basic wash is concentrated to dryness to afford 1-methyl-pyrrolo[2,3-c]pyridine-5-carboxylic acid (C178) (78% yield). HRMS (FAB) calculated for C₉H₈N₂O₂+H: 177.0664, found 177.0672 (M+H)⁺.

Example 7 is obtained as a yellow solid (54% yield) using acid C178 according to Method B with non-critical changes. HRMS (FAB) calculated for C₁₆H₂₀N₄O+H: 285.1715, found 285.1713 (M+H)⁺.

### EXAMPLE 8

### N-[(3S)-1-azabicyclo[2.2.2]oct-3-yl]-1-methyl-1H-pyrrolo[2,3-c]pyridine-5-carboxamide dihydrochloride

Example 8 can be prepared using Method B, making non-critical changes and using (S)-3-aminoquinuclidine free base.

### EXAMPLE 9

### N-[(3R)-1-azabicyclo[2.2.2]oct-3-yl]-3-chlorofuro[2,3-c]pyridine-5-carboxamide dihydrochloride

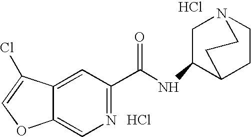

Furo[2,3-c]pyridin-5-ylmethanol (7.70 g, 51.63 mmol) is dissolved in pyridine (45 mL), treated with acetic anhydride (14.36 mL, 154.9 mmol) and stirred for 18 h at rt. The pyridine is removed in vacuo and the resulting residue dissolved in EtOAc (200 mL), washed with 50% saturated sodium bicarbonate (4×90 mL), dried (MgSO₄)and concentrated in vacuo to afford 9.32 g (94%) of furo[2,3-c]pyridin-5-ylmethyl acetate as a yellow oil. MS (EI) m/z: 191 (M⁺), 277, 148, 119, 118, 86, 84, 77, 63, 51, 50.

Furo[2,3-c]pyridin-5-ylmethyl acetate (956 mg, 5 mmol) is dissolved in CH₂Cl₂ (40 mL) and cooled to 0° C. Chlorine gas is bubbled through the solution for 15 min, the cooling bath is immediately removed and the mixture stirred for 2 h. The mixture is re-cooled to 0° C., saturated with chlorine gas, the cooling bath removed and the solution warmed to rt. The solution is layered with saturated NaHCO₃ (20 mL), stirred gently for 2 h then stirred vigorously for 15 min. The mixture is diluted with saturated NaHCO₃ (50 mL), extracted with CH₂Cl₂ (1×40 mL then 1×20 mL), dried over K₂CO₃ and concentrated to a volume of 20 mL under a stream of nitrogen. The solution is diluted with EtOH (35 mL), treated with K₂CO₃ (4.09 g, 29.6 mmol) and stirred for 18 h at rt. Water (7 mL) is added and the mixture stirred for 2 days. The mixture is concentrated to dryness, partitioned between 50% saturated NaCl (50 mL) and CH₂Cl₂ (4×50 mL), dried over K₂CO₃ and concentrated in vacuo to a brown solid (833 mg). The crude material is chromatographed over a standard 40 g Biotage column, eluting with 50% EtOAc/hexane. The appropriate fractions are combined and concentrated to afford 624 mg (68%) of (3-chlorofuro[2,3-c]pyridin-5-yl)methanol as a yellow oil. ¹H NMR (DMSO-d₆): δ 4.69, 5.56, 7.69, 8.55, 8.93 ppm.

Oxalyl chloride (231 μL, 2.6 mmol) is combined with CH₂Cl₂ (10 mL), cooled to −78° C., treated dropwise with DMSO (373 μL, 5.3 mmol) and stirred for 20 min. The cooled solution is treated dropwise with a solution of (3-chlorofuro[2,3-c]pyridin-5-yl)methanol (420 mg, 2.3 mmol) in THF (5 mL)/CH₂Cl₂ (5 mL), stirred for 1 h, then treated dropwise with Et₃N (1.59 mL, 11.45 mmol). The mixture is stirred for 30 min at −78° C., then 30 min at 0° C. The mixture is washed with saturated NaHCO₃ (20 mL) and the organics dried over K₂CO₃ and concentrated in vacuo to a yellow solid (410 mg). The crude material is chromatographed over 20 g slurry-packed silica gel, eluting with 15% EtOAc/hexane. The appropriate fractions are combined and concentrated in vacuo to afford 322 mg (77%) of 3-chlorofuro[2,3-c]pyridine-5-carbaldehyde as a white solid. ¹H NMR (CDCl₃): δ 7.89, 8.33, 9.02, 10.18 ppm.

3-Chlorofuro[2,3-c]pyridine-5-carbaldehyde (317 mg, 1.74 mmol) is dissolved in THF (10 mL)/t-BuOH (5 mL)/H₂O (5 mL), treated with a single portion of sodium chlorite (592 mg, 5.24 mmol) and KH₂PO₄ (473 mg, 3.48 mmol) and stirred at rt for 18 h. The reaction mixture is concentrated in vacuo to dryness, suspended in water (10 mL), acidified to pH 3.5 with concentrated HCl and stirred at rt for 2 h. The resulting solid is filtered, washed with water and dried in a vacuum oven at 40° C. for 18 h to afford 364 mg of 3-chlorofuro[2,3-c]pyridine-5-carboxylic acid as a white solid. MS (EI) m/z: 197 (M⁺).

Example 9 is obtained using 3-chlorofuro[2,3-c]pyridine-5-carboxylic acid accoding to Method B making non-critical changes to afford 101 mg of a white solid. MS (EI) m/z: 305 (M⁺).

### EXAMPLE 10

### N-[(3S)-1-azabicyclo[2.2.2]oct-3-yl]-3-chlorofuro[2,3-c]pyridine-5-carboxamide

Example 10 can be prepared using Method B, making non-critical changes and using (S)-3-aminoquinuclidine free base.

### EXAMPLE 11

### N-[(3R)-1-azabicyclo[2.2.2]oct-3-yl]-3-bromofuro[2,3-c]pyridine-5-carboxamide

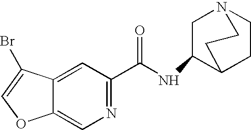

Furo[2,3-c]pyridin-5-ylmethyl acetate (5.17 g, 27.05 mmol) is dissolved in CH₂Cl₂ (130 mL), layered with saturated NaHCO₃ (220 mL), treated with Br₂ (8.36 mL, 162.3 mmol) and stirred very slowly for 4.5 h at rt. The mixture is stirred vigorously for 30 min, is diluted with CH₂Cl₂ (100 mL) and the layers separated. The aqueous layer is extracted with CH₂Cl₂ (2×100 mL) and the combined organics are concentrated to a small volume under a stream of nitrogen. The solution is diluted with EtOH (200 mL), treated with K₂CO₃ (22.13 g, 160.1 mmol) and stirred for 2.5 days at rt. The mixture is concentrated to dryness, partitioned between 50% saturated NaCl (200 mL) and CH₂Cl₂ (5×200 mL), dried over Na₂SO₄ and concentrated in vacuo to a yellow solid (6.07 g). The crude material is adsorbed onto silica gel (12 g) and chromatographed over 250 g slurry-packed silica gel, eluting with a gradient of 50% EtOAc/hexane to 100% EtOAc. The appropriate fractions are combined and concentrated in vacuo to afford 5.02 g (81%) of (3-bromofuro[2,3-c]pyridin-5-yl)methanol as a white solid. MS (EI) m/z: 227 (M⁺).

Oxalyl chloride (1.77 mL, 20.1 mmol) is combined with CH₂Cl₂ (60 mL) in a dried flask under nitrogen, cooled to −78° C., treated dropwise with DMSO (2.86 mL, 40.25 mmol) and stirred for 20 min. The cooled solution is treated drop-wise with a solution of (3-bromofuro[2,3-c]pyridin-5-yl)methanol (4.0 mg, 17.5 mmol) in THF (50 L), stirred for 1 h, then treated drop-wise with Et₃N (12.2 mL, 87.5 mmol). The mixture is stirred for 30 min at −78° C., then 30 min at 0C. The mixture is washed with saturated NaHCO₃ (120 mL) and the organics dried over K₂CO₃ and concentrated in vacuo to a dark yellow solid (3.91 g). The crude material is chromatographed over 150 g slurry-packed silica gel, eluting with 30% EtOAc/hexane. The appropriate fractions are combined and concentrated in vacuo to afford 3.93 g (99%) of 3-bromofuro[2,3-c]pyridine-5-carbaldehyde as a white solid. MS (EI) m/z: 225 (M⁺).

3-Bromofuro[2,3-c]pyridine-5-carbaldehyde (3.26 g, 14.42 mmol) is dissolved in THF (100 mL)/t-BuOH (50 mL)/H₂O (50 mL), treated with a single portion of NaOCl₂ (4.89 g, 43.3 mmol) and KH₂PO₄ (3.92 g, 28.8 mmol) and stirred at rt for 18 h. The white solid is collected via filtration and the filtrate is concentrated in vacuo to dryness. The residue is suspended in water (25 mL), acidified to pH 2 with concentrated HCl and the resulting solid collected via filtration. The collected solids are dried in a vacuum oven at 50° C. for 18 h and combined to afford 3.52 g (99%) of 3-bromofuro[2,3-c]pyridine-5-carboxylic acid as a white solid. MS (EI) m/z: 241 (M⁺).

Example 11 is obtained using 3-bromofuro[2,3-c]pyridine-5-carboxylic acid according to Method B making non-critical changes to afford 670 mg (96% yield) of a white solid. MS (EI) m/z: 335 (M⁺).

### EXAMPLE 12

### N-[(3S)-1-azabicyclo[2.2.2]oct-3-yl]-3-bromofuro[2,3-c]pyridine-5-carboxamide

Example 12 can be prepared using Method B, making non-critical changes and using (S)-3-aminoquinuclidine free base.

### EXAMPLE 13

### N-[(3R)-1-azabicyclo[2.2.2]oct-3-yl]-3-bromothieno[2,3-c]pyridine-5-carboxamide

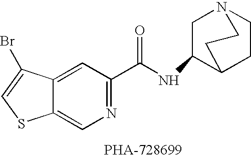

C154 (630 mg, 3.3 mmol) is dissolved in 20 ml CH₂Cl₂. The solution is treated with Br₂ (1.1 ml, 20 mmol), is layered with 20 ml saturated NaHCO₃, and the two-phase mixture is agitated gently for 2 h. The reaction is stirred vigorously for 30 min, the layers are separated, and the organic layer is dried over anhydrous K₂CO₃. The organic layer is concentrated to a dark tan solid. The solid is dissolved in 20 ml 10% MeOH/CH₂Cl₂, is adsorbed onto 2 g silica gel (230-400 mesh), and chromatographed over 25 g silica gel (230-400 mesh) eluting with 65% EtOAc/hexane. The appropriate fractions are combined and concentrated to afford 635 mg (71%) of methyl-3-bromothieno[2,3-c]pyridine-5-carboxylate as a tan solid. ¹H NMR (CDCl₃) δ 4.09, 7.82, 8.59, 9.25 ppm.

Methyl-3-bromothieno[2,3-c]pyridine-5-carboxylate (635 mg, 2.33 mmol) is combined with 25 ml MeOH. The mixture is treated with 2N NaOH (3 ml, 6 mmol) and 3 ml H₂O and the reaction is stirred 4 h at rt. The volatiles are removed in vacuo and the residue is combined with 5 ml H₂O. The pH of the mixture is adjusted to 3.5 with 10% aqueous HCl. The tan precipitate is collected, washed with water, and is dried in vacuo at 50° C. to afford 475 mg (79%) of 3-bromothieno[2,3-c]pyridine-5-carboxylic acid as a tan solid. MS (ESI): 257.9.

Example 13 is obtained using 3-bromothieno[2,3-c]pyridine-5-carboxylic acid according to Method B to afford 240 mg (91%) of an off-white solid. MS (EI) m/z: 365 (M⁺).

### EXAMPLE 14

### N-[(3 S)-1-azabicyclo[2.2.2]oct-3-yl]-3-bromothieno[2,3-c]pyridine-5-carboxamide

Example 14 can be prepared using Method B, making non-critical changes and using (S)-3-aminoquinuclidine free base.

### EXAMPLE 15

### N-[(3R)-1-azabicyclo[2.2.2]oct-3-yl]-3-isopropyl-1-benzofuran-5-carboxamide hydrochloride

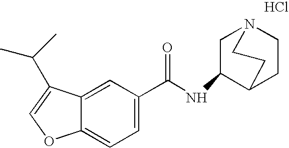

Methyl 4-hydroxy-3-iodobenzoate (6.0 g, 21.5 mmol) is dissolved in DMF (35 ml) in a dry flask under nitrogen and cooled to 0° C. 60% Sodium hydride (860 mg, 21.5 mmol) is added portionwise, and the reaction is stirred 1 h, allowing the ice bath to expire. The mixture is then treated with 1-chloro-3-methyl-2-butene (2.67 ml, 23.7 mmol) and sodium iodide (323 mg, 2.15 mmol), and the reaction is stirred 18 h at rt. The mixture is diluted with EtOAc (150 ml) and washed with 1:1 saturated NaCl/NaHCO₃ (1×100 ml). The organic layer is dried with MgSO₄ and concentrated to an oil. The crude material is chromatographed over 700 g slurry-packed silica gel, eluting with 15% EtOAc/hexane. The appropriate fractions are collected and concentrated to afford 5.13 g of a pale oil. The oil is then dissolved in DMF (40 ml) and treated successively with palladium acetate (165 mg, 0.74 mmol), sodium carbonate (3.9 g, 36.8 mmol), sodium formate (1.0 g, 14.7 mmol), and tetra N-butyl ammonium chloride (4.5 g, 16.2 mmol). The mixture is stirred 2 days at 80° C. The reaction is poured onto EtOAc (200 ml) and washed with 50% saturated brine (3×75 ml) and 5% HCl (1×75 ml). The organic layer is dried (MgSO₄), filtered, and concentrated to a brown oil. The crude material is chromatographed over 250 g slurry-packed silica gel, eluting with 10% EtOAc/hexane. The appropriate fractions are collected and concentrated to afford 1.33 g (28% over 2 steps) of methyl 3-isopropyl-1-benzofuran-5-carboxylate as a mobile oil. HRMS (FAB) calcd for C₁₃H₁₄O₃+H: 219.1021, found 219.1021 (M+H)⁺.

Methyl 3-isopropyl-1-benzofuran-5-carboxylate (1.20 g, 5.51 mmol) is dissolved in MeOH (20 ml) and H₂O (4 ml). 2N NaOH (3.3 ml, 6.6 mmol) is added dropwise, and the reaction is stirred 2 days. Slight heating at 40° C. is required for 4 h. Volatiles are removed in vacuo, and the residue is dissolved in H₂O (10 ml). Concentrated HCl is used to adjust the pH to 3, and the resulting precipitate is isolated via filtration and dried overnight to afford 1.08 g (97%) of 3-isopropyl-1-benzofuran-5-carboxylic acid as a white solid. MS (ESI) for C₁₂H₁₂O₃ m/z: 203.0 (M−H)⁻.

Example 15 is obtained in 90% yield as a white solid using Method B, making non-critical changes. HRMS (FAB) calcd for C₁₉H₂₄N₂O₂+H: 313.1916, found 313.1913 (M+H)⁺.

### EXAMPLE 16

### N-[(3S)-1-azabicyclo[2.2.2]oct-3-yl]-3-isopropyl-1-benzofuran-5-carboxamide hydrochloride

Example 16 can be prepared using Method B, making non-critical changes and using (S)-3-aminoquinuclidine free base.

### EXAMPLE 17

### N-[(1S, 2R, 4R)-7-azabicyclo[2.2.1]hept-2-yl]-3-isopropyl-1-benzofuran-5-carboxamide hydrochloride

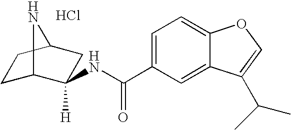

Example 17 is obtained in 73% yield using Method B, making non-critical changes by coupling 3-isopropyl-1-benzofuran-5-carboxylic acid with tert-butyl (2R)-2-amino-7-azabicyclo[2.2.1]heptane-7-carboxylate, and removing the carbonate with methonolic HCl. HRMS (FAB) calcd for C₁₈H₂₂N₂O₂+H: 299.1759, found 299.1754 (M+H)⁺.

### EXAMPLE 18

### N-[(3R)-1-azabicyclo[2.2.2]oct-3-yl]-1-methyl-1H-indole-5-carboxamide.fumarate

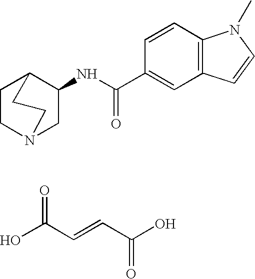

To a stirred suspension of 0.99 g (24.8 mmol) of sodium hydride (60% oil dispersion), which had been previously washed 3× with hexanes, in anhydrous DMF (50 mL) is added 1H-indole-5-carboxylic acid (2.0 g, 12.4 mmol). The mixture is stirred at rt for 30 min and methyl iodide (3.09 mL, 49.7 mmol) is added. The mixture is stirred overnight and diluted with water, extracted with EtOAc (3×). The combined organic layers are washed with water and brine, dried over anhydrous sodium sulfate, filtered and concentrated in vacuo. The crude product is purified by flash chromatography on silica gel. Elution with hexanes-EtOAc (90:10) gives methyl 1-methyl-1H-indole-5-carboxylate as a white solid (1.32 g, 56%): ¹H NMR (400 MHz, CDCl₃) δ 8.44, 7.97, 7.37, 7.16, 6.63, 3.97, 3.87.

To a stirred solution of methyl 1-methyl-1H-indole-5-carboxylate (500 mg, 2.65 mmol) in MeOH (5 mL) is added sodium hydroxide (20 mL of a 2.5% aqueous solution). The mixture is heated at 80° C. for 1.5 h and MeOH is removed in vacuo. The remaining aqueous solution is acidified with 1 N aqueous HCl to pH=2. The resulting precipitate is collected by filtration, washed with water and dried in vacuo to afford 1-methyl-1H-indole-5-carboxylic acid as a white solid (437 mg, 94%): ¹H NMR (400 MHz, DMSO-d₆) δ 12.44, 8.23, 7.75, 7.50, 7.44, 6.57, 3.83.

The free base of Example 18 is obtained in 100% yield using Method B, making non-critical changes.

To a stirred solution of the free base (408 mg, 1.43 mmol) in MeOH (5 mL) is added a warm solution of fumaric acid (167 mg, 1.43 mmol) in MeOH (5 mL). The mixture is stirred for 10 min at 50° C. The solvent is removed in vacuo, and the remaining residue is diluted with acetone (5 mL) and water (0.5 mL). The mixture is stirred overnight at rt. The solid is collected by filtration, washed with acetone, and dried under high vacuum overnight to give 509 mg (89%) of Example 18 as a white solid: ¹H NMR (400 MHz, MeOH-d₄) δ 8.17, 7.73, 7.47, 7.30, 6.71, 6.58, 4.49-4.44, 3.88-3.82, 3.87, 3.49-3.25, 2.40-2.37, 2.32-2.24, 2.14-2.09, 1.99-1.91.

### EXAMPLE 19

### N-[(3R)-1-azabicyclo[2.2.2]oct-3-yl]-6-bromopyrrolo[1,2-a]pyrazine-3-carboxamide fumerate

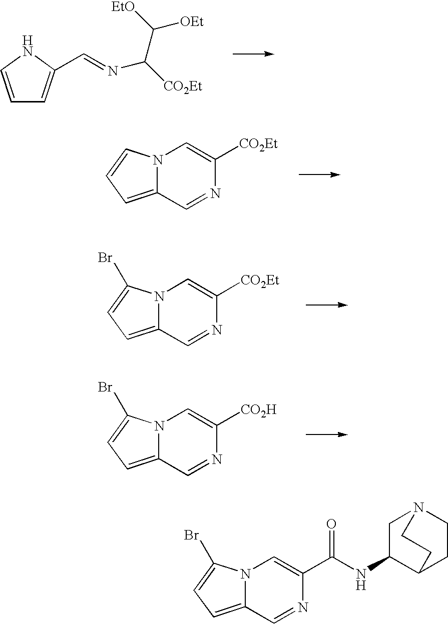

To a hot (65° C.) solution of TFA (44 mL, 510 mmol) and phosphorus oxychloride (39.0 g, 140 mmol) is added drop-wise a solution of ethyl 3-ethoxy-O-ethyl-N-(1H-pyrrol-2-ylmethylene)serinate (Dekhane, M; Potier, P; Dodd, R. H. Tetrahedron, 49, 1993, 8139-46) (9.6 g, 28.0 mmol) in anhydrous 1,2-dichloroethane (200 mL). The black mixture is allowed to stir at 65° C. for 18 hr at which point it is cooled to rt and neutralized with sat. NaHCO₃ and solid NaHCO₃ to pH ˜9. The phases are separated, and the basic phase extracted with EtOAc (4×100 mL). The organic phases are combined, washed with brine, dried over Na₂SO₄, filtered, and concentrated to give a black oil that is purified with silica gel chromatography (35% EtOAc/heptanes to 50% over several liters) to give a light brown solid for ethyl pyrrolo[1,2-a]pyrazine-3-carboxylate. Yield 24%. HRMS (FAB) calcd for C₁₀H₁₀N₂O₂+H 191.0820, found 191.0823.

To a solution of ethyl pyrrolo[1,2-a]pyrazine-3-carboxylate (0.10 g, 0.54 mmol) in CH₂Cl₂ (10 mL) protected from light is added N-bromosuccinimide (0.09 g, 0.54 mmol). After 10 min, the solvent is removed in vacou and the residue purified with preparatory chromatography to give ethyl 6-bromopyrrolo[1,2-a]pyrazine-3-carboxylate in yield 57%. MS (ESI+) for C₁₀H₉BrN₂O₂ m/z 269.0 (M+H)⁺.

To a solution of ethyl 6-bromopyrrolo[1,2-a]pyrazine-3-carboxylate (1.56 g, 5.80 mmol) in EtOH (170 mL) is added water (70 mL) followed by potassium hydroxide (3.2 g, 58.0 mmol). After 20 min, conc. HCl is added until the pH is approximately 1-2. The mixture is concentrated to dryness under reduced pressure, and the resulting mixture of 6-bromopyrrolo[1,2-a]pyrazine-3-carboxylic acid hydrochloride and potassium chloride is utilized without purification. MS (ESI+) for C₈H₅BrN₂O₂ m/z 241.1 (M+H)⁺.

To a suspension of 6-bromopyrrolo[1,2-a]pyrazine-3-carboxylic acid hydrochloride (1.67 mmols), (R)-3-aminoquinulidine dihydrochloride (0.34 g, 1.67 mmol), DIEA (1.5 mL, 8.35 mmols) in DMF (20 mL) and THF (10 mL) is added N-[(dimethylamino)-1H-1,2,3-triazolo[4,5-b]pyridin-1-ylmethylene]-N-methyl-methanaminium hexafluorophosphate N-oxide (0.64 g, 1.67 mmol). The resulting suspension is stirred for 16 h at which time it is concentrated to dryness under reduced pressure. The resulting material is absorbed to silica gel and purified with silica gel chromatography (9% MeOH/1% NH₃OH/CH₂Cl₂ as the eluent). Example 19 is obtained in 45% yield following procedures used in Example 18, making non-critical changes. HRMS (FAB) calcd. for C₁₅H₁₇BrN₄O+H 349.0664, found 349.0647.

### EXAMPLE 20

### N-[(3R)-1-azabicyclo[2.2.2]oct-3-yl]-6-ethynylpyrrolo[1,2-a]pyrazine-3-carboxamide tartrate

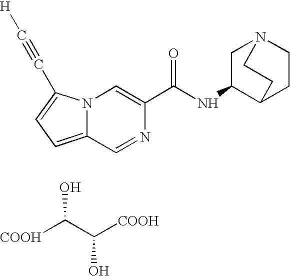

To a degassed solution of N-[(3R)-1-azabicyclo[2.2.2]oct-3-yl]-6-bromopyrrolo[1,2-a]pyrazine-3-carboxamide (0.59 g, 1.7 mmol), TEA (5.8 mL, 42.2 mmol) in dioxane (10 mL) is added copper_{(I)} iodide (0.09 g, 0.50 mmol), (triisopropylsilyl) acetylene (1.54 g, 8.5 mmol), and dichlorobis(triphenylphosphine)palladium(II) (0.12 g, 0.17 mmol). The resulting mixture is stirred at 80° C. for 17.5 h, cooled to rt, and concentrated to dryness. The residue is taken up in CHCl₃ and washed with a solution of 1:1 NH₄OH/brine (3×50 mL), dried over Na₂SO₄, filtered, and concentrated to dryness. The resulting material is purified with preparative HPLC (reversed phase C18, gradient 40% to 25% (5 mM (NH₄)₂CO₃ (aqueous) in CH₃CN) to give a colored oil. Yield 60%. HRMS (FAB) calcd for C₂₆H₃₈N₄OSi+H: 451.2893, found 451.2872.

To a solution of N-[(3R)-1-azabicyclo[2.2.2]oct-3-yl]-6-[(triisopropylsilyl)ethynyl] pyrrolo[1,2-a]pyrazine-3-carboxamide (0.45 g, 1.0 mmol) in THF (40 mL) is added a 1.0 M solution of tetrabutylammonium fluoride in THF (4.0 mL). The resulting solution is allowed to stir for 20 min at which point it is concentrated to dryness and absorbed to silica gel and purified with silica gel chromatography (5% MeOH/1% NH₃OH/CH₂Cl₂ to 10% as the eluent)

The compound is dissolved in EtOH and d-tartaric acid is added (1 eq) and the resulting mixture is crystallized from EtOH/Et₂O to give a pale brown solid. Yield 98%. HRMS (FAB) calcd for C₁₇H₁₈N₄O+H 295.1559, found 295.1566.

### EXAMPLE 21

### N-[(3S)-1-azabicyclo[2.2.2]oct-3-yl]-1-benzofuran-5-carboxamide-(2E)-but-2-enedioic acid

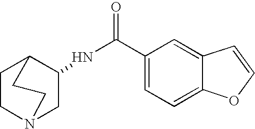

See: Dunn, J. P.; Ackerman, N. A.; Tomolois, A. J. J. Med. Chem. 1986, 29, 2326. This procedure was used without significant changes to afford 1-(2,3-dihydrobenzofuran-5-yl)ethanone 1 in similar yield (82%) and of similar purity (95%): ¹H NMR (400 MHz, CDCl₃) δ 7.89, 7.83, 6.84, 4.70, 3.29, 2.58.

A mixture of 1 (4.0 g, 25 mmol) and sodium hypochlorite [160 mL of a 6.0% aqueous solution, (Clorox brand of bleach)] at 55° C. is stirred for 1 h. The mixture (now homogeneous) is cooled to room temperature and solid sodium bisulfite is added until a clear color persists. Hydrochloric acid (80 mL of a 1.0 N aqueous solution) is added, followed by extraction with ethyl acetate. The organic layer is washed with brine, dried over anhydrous magnesium sulfate, filtered, and concentrated in vacuo to afford 3.93 g (97%) of 2,3-dihydrobenzofuran-5-carboxylic acid 2 as a white solid: ¹H NMR (400 MHz, CDCl₃) δ 11.0-10.3, 8.00, 6.87, 4.72, 3.31.

To a stirred solution of 2 (3.96 g, 24.1 mmol) in MeOH (200 mL) is added concentrated sulfuric acid (0.5 mL). The mixture is heated to reflux for 24 h. The mixture is cooled to room temperature, followed by the addition of solid sodium bicarbonate. The reaction mixture is concentrated in vacuo and the remaining residue is partitioned between ethyl acetate and water. The aqueous layer is extracted with ethyl acetate, and the combined organic layers are dried over anhydrous magnesium sulfate, filtered and concentrated in vacuo to afford 4.22 g (98%) of methyl 2,3-dihydrobenzofuran-5-carboxylate 3 as a white solid: ¹H NMR (400 MHz, CDCl₃) δ 7.93-7.89, 6.82, 4.69, 3.86, 3.28.

To a stirred solution of 3 (4.2 g, 24 mmol) in anhydrous p-dioxane (150 mL) under argon atmosphere is added 2,3-dichloro-5,6-dicyano-1,4-benzoquinone (6.42 g, 28 mmol). The mixture is heated to reflux for 24 h, followed by cooling to room temperature. The reaction mixture is partitioned between ether and ½ saturated aqueous sodium carbonate solution. The organic layer is extracted several times with ½ saturated aqueous sodium carbonate solution. The organic layer is washed with water, dried over anhydrous magnesium sulfate, filtered, and concentrated in vacuo to give 4.2 g (92%) of a mixture (1:3) of recovered starting material 3 and methyl benzofuran-5-carboxylate 4, respectively. The crude product is purified by preparative HPLC using a Chiralcel OJ column. Elution with heptane-iso-propyl alcohol, (80:20, flow rate=70 mL/min) gave 0.75 g (18%) of 3 as a white solid and 2.5 g (61%) of 4 as a white solid. Benzofuran 4: ¹H NMR (400 MHz, CDCl₃) δ 8.40, 8.07, 7.73, 7.57, 6.89, 3.99.

A stirred mixture of 4 (1.3 g, 7.38 mmol) in methanol (51 mL) and sodium hydroxide (41 mL of a 5% aqueous solution) is heated to 65° C. for 4 h. The mixture is cooled to room temperature, and the methanol is removed in vacuo. The remaining aqueous layer is extracted with methylene chloride. The methylene chloride layer is discarded, and the aqueous layer is acidified to pH=1 with concentrated hydrochloric acid. The aqueous layer is extracted with chloroform. The organic layer is washed with water, dried over anhydrous magnesium sulfate, filtered and concentrated in vacuo to afford 1.2 g (98%) of benzofuran-5-carboxylic acid 5 as a white solid: H NMR (400 MHz, DMSO-d₆) δ 12.9, 8.30, 8.11, 7.92, 7.69, 7.09.

The free base of Example 21 is obtained in 94% yield as a white solid using Method B, making non-critical changes.

The free base 3.3 g (12.2 mmol) is dissolved in methanol (20 mL) and fumaric acid (3.5 g, 12.2 mmol) is added. The mixture is warmed to 50° C. for 30 min. The solvent is removed in vacuo. The remaining residue is diluted with water (20 mL), and recrystallized from methanol and diethyl ether to give 1.6 g of Example 21 as a white solid. Anal. Calcd for C₁₆H₁₈N₂O₃.C₄H₄O₄.1.1 H₂O: C, 59.14; H, 6.00; N, 6.90. Found: C, 58.84; H, 5.92; N, 6.62.

### Materials and Methods for Determining α7 nAChR Agonist Activity & 5-HT₃ Antagonist Activity

Cell-Based Assay for Measuring the EC₅₀ of α7 nAChR Agonists

Construction and Expression of the α7-5HT₃ Receptor:

The cDNA encoding the N-terminal 201 amino acids from the human α7 nAChR that contain the ligand binding domain of the ion channel was fused to the cDNA encoding the pore forming region of the mouse 5HT₃ receptor as described by Eisele J L, et al., Chimaeric nicotinic-serotonergic receptor combines distinct ligand binding and channel specificities, Nature (1993), Dec. 2;366(6454):479-83, and modified by Groppi, et al., WO 00/73431. The chimeric α7-5HT₃ ion channel was inserted into pGS175 and pGS179 which contain the resistance genes for G-418 and hygromycin B, respectively. Both plasmids were simultaneously transfected into SH-EP1 cells and cell lines were selected that were resistant to both G-418 and hyrgromycin B. Cell lines expressing the chimeric ion channel were identified by their ability to bind fluorescent α-bungarotoxin on their cell surface. The cells with the highest amount of fluorescent α-bungarotoxin binding were isolated using a Fluorescent Activated Cell Sorter (FACS). Cell lines that stably expressed the chimeric α7-5HT₃ were identified by measuring fluorescent α-bungarotoxin binding after growing the cells in minimal essential medium containing nonessential amino acids supplemented with 10% fetal bovine serum, L-glutamine, 100 units/ml penicillin/streptomycin, 250 ng/mg fungizone, 400 μg/ml hygromycin B, and 400 μg/ml G-418 at 37° C. with 6% CO₂ in a standard mammalian cell incubator for at least 4 weeks in continuous culture.

Assay of the Activity of the Chimeric α7-5HT₃ Receptor

To assay the activity of the α7-5HT₃ ion channel, cells expressing the channel were plated into each well of either a 96 or 384 well dish (Corning #3614) and grown to confluence prior to assay. On the day of the assay, the cells were loaded with a 1:1 mixture of 2 mM Calcium Green 1, AM (Molecular Probes) dissolved in anhydrous DMSO and 20% pluronic F-127 (Molecular Probes). This solution was added directly to the growth media of each well to achieve a final concentration 2 μM. The cells were incubated with the dye for 60 min at 37° C. and is washed with a modified version of Earle's balanced salt solution (MMEBSS) as described in WO 00/73431. The ion conditions of the MMEBSS was adjusted to maximize the flux of calcium ion through the chimeric α7-5HT₃ ion channel as described in WO 00/73431. The activity of compounds on the chimeric α7-5HT₃ ion channel was analyzed on FLIPR. The instrument was set up with an excitation wavelength of 488 nanometers using 500 milliwatts of power. Fluorescent emission was measured above 525 nanometers with an appropriate F-stop to maintain a maximal signal to noise ratio. Agonist activity of each compound was measured by directly adding the compound to cells expressing the chimeric α7-5HT₃ ion channel and measuring the resulting increase in intracellular calcium that is caused by the agonist-induced activation of the chimeric ion channel. The assay is quantitative such that concentration-dependent increase in intracelluar calcium is measured as concentration-dependent change in Calcium Green fluorescence. The effective concentration needed for a compound to cause a 50% maximal increase in intracellular calcium is termed the EC₅₀.

Binding Constants:

Another way for measuring α7 nAChR agonist activity is to determine binding constants of a potential agonist in a competition binding assay. For α7 nAChR agonists, there is good correlation between functional EC₅₀ values using the chimeric α7-5HT₃ ion channel as a drug target and binding affinity of compounds to the endogenous α7 nAChR.

Membrane Preparation

Male Sprague-Dawley rats (300-350 g) are sacrificed by decapitation and the brains (whole brain minus cerebellum) are dissected quickly, weighed and homogenized in 9 volumes/g wet weight of ice-cold 0.32 M sucrose using a rotating pestle on setting 50 (10 up and down strokes). The homogenate is centrifuged at 1,000×g for 10 min at 4° C. The supernatant is collected and centrifuged at 20,000×g for 20 min at 4° C. The resulting pellet is resuspended to a protein concentration of 1-8 mg/mL. Aliquots of 5 mL homogenate are frozen at −80° C. until needed for the assay. On the day of the assay, aliquots are thawed at rt and diluted with Kreb's—20 mM Hepes buffer pH 7.0 (at rt) containing 4.16 mM NaHCO₃, 0.44 mM KH₂PO₄, 127 mM NaCl, 5.36 mM KCl, 1.26 mM CaCl₂, and 0.98 mM MgCl₂, so that 25-150 μg protein are added per test tube. Proteins are determined by the Bradford method (Bradford, M. M., Anal. Biochem., 72, 248-254, 1976) using bovine serum albumin as the standard.

Binding Assay

For saturation studies, 0.4 mL homogenate are added to test tubes containing buffer and various concentrations of radioligand, and are incubated in a final volume of 0.5 mL for 1 hour at 25° C. Nonspecific binding was determined in tissues incubated in parallel in the presence of 0.05 mls MLA for a final concentration of 1 μM, added before the radioligand. In competition studies, drugs are added in increasing concentrations to the test tubes before addition of 0.05 mls [³H]-MLA for a final concentration 3.0 to 4.0 nM. The incubations are terminated by rapid vacuum filtration through Whatman GF/B glass filter paper mounted on a 48 well Brandel cell harvester. Filters are pre-soaked in 50 mM Tris HCl pH 7.0-0.05% polyethylenimine. The filters are rapidly washed two times with 5 mL aliquots of cold 0.9% saline and counted for radioactivity by liquid scintillation spectrometry.

Data Analysis

In competition binding studies, the inhibition constant (Ki) was calculated from the concentration dependent inhibition of [³H]-MLA binding obtained from non-linear regression fitting program according to the Cheng-Prusoff equation (Cheng, Y. C. and Prussoff, W. H., Biochem. Pharmacol., 22, p. 3099-3108, 1973). Hill coefficients were obtained using non-linear regression (GraphPad Prism sigmoidal dose-response with variable slope).

Methods for determing 5-HT₃ antagonist activity of compounds is well known to those skilled in the art and can be used to identify the compounds of the present invention as 5-HT₃ antagonists.

What is claimed:

1. A compound of Formula I:
Azabicyclo-N(H)—C(═O)—W⁰ Formula I
wherein Azabicyclo is 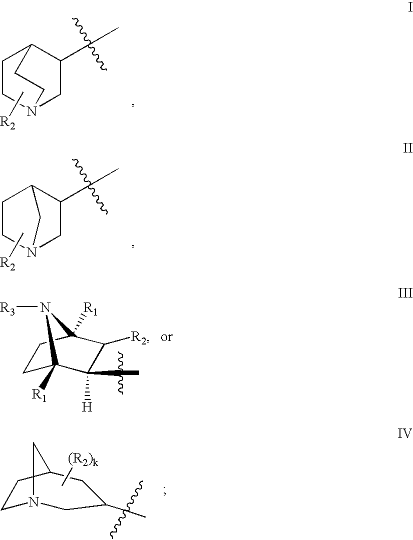
Each R₁ is independently H, alkyl, or substituted alkyl;
Each R₂ is independently H, alkyl, or substituted alkyl;
k is 1 or 2, provided that one R₂ is other than H when k is 2;
R₃ is H, alkyl, or an amino protecting group;
W⁰ is 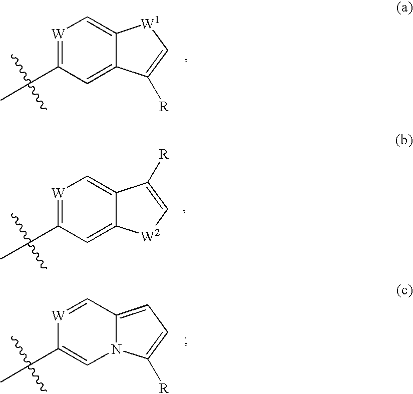
W is CH or N;
W¹ is O, N(R₄), N(C(O)R₄), or S;
W² is O, N(R₄), N(C(O)R₄), or S;
R is H, F, Cl, Br, I, alkyl, substituted alkyl, or alkynyl;
Each R₄ is independently H or alkyl optionally substituted where valency allows with up to 3 substituents independently selected from —OH, —CN, NH₂, —NO₂, —CF₃, F, Cl, Br, or I;
and pharmaceutically acceptable salts thereof.

2. The compound of claim 1, wherein R₂ is lower alkyl or substituted lower alkyl.

3. The compound of claim 2, wherein Azabicyclo is I.

4. The compound of claim 2, wherein Azabicyclo is II.

5. The compound of claim 2, wherein Azabicyclo is III.

6. The compound of claim 5, wherein each R₁ is independently H, lower alkyl, or lower substituted alkyl.

7. The compound of claim 6, wherein R₃ is H, or lower alkyl.

8. The compound of claim 6, wherein R₃ is an amino protecting group.

9. The compound of claim 2, wherein Azabicyclo is IV.

10. The compound of claim 9, wherein k is 1.

11. The compound of claim 2, wherein R is F, Cl, Br, I, lower alkyl, lower substituted alkyl, or lower alkynyl.

12. The compound of claim 11, wherein W is CH.

13. The compound of claim 12, wherein the compound is
N-[(3R)-1-azabicyclo[2.2.2]oct-3-yl]-3-bromo-1-benzofuran-5-carboxamide
N-[(3S)-1-azabicyclo[2.2.2]oct-3-yl]-3-bromo-1-benzofuran-5-carboxamide
N-[(3R)-1-azabicyclo[2.2.2]oct-3-yl]-3-isopropyl-1-benzofuran-5-carboxamide
N-[(3S)-1-azabicyclo[2.2.2]oct-3-yl]-3-isopropyl-1-benzofuran-5-carboxamide
N-[(1S, 2R, 4R)-7-azabicyclo[2.2.1]hept-2-yl]-3-isopropyl-1-benzofuran-5-carboxamide, or a pharmaceutically acceptable salt thereof.

14. The compound of claim 11, wherein W is N.

15. The compound of claim 14, wherein the compound is
N-[(3R)-1-azabicyclo[2.2.2]oct-3-yl]thieno[3,2-c]pyridine-6-carboxamide
N-[(3S)-1-azabicyclo[2.2.2]oct-3-yl]thieno[3,2-c]pyridine-6-carboxamide
N-[(3R)-1-azabicyclo[2.2.2]oct-3-yl]-3-chlorofuro[2,3-c]pyridine-5-carboxamide
N-[(3S)-1-azabicyclo[2.2.2]oct-3-yl]-3-chlorofuro[2,3-c]pyridine-5-carboxamide
N-[(3R)-1-azabicyclo[2.2.2]oct-3-yl]-3-bromofuro[2,3-c]pyridine-5-carboxamide
N-[(3S)-1-azabicyclo[2.2.2]oct-3-yl]-3-bromofuro[2,3-c]pyridine-5-carboxamide
N-[(3R)-1-azabicyclo[2.2.2]oct-3-yl]-3-bromothieno[2,3-c]pyridine-5-carboxamide
N-[(3R)-1-azabicyclo[2.2.2]oct-3-yl]-6-bromopyrrolo[1,2-a]pyrazine-3-carboxamide
N-[(3R)-1-azabicyclo[2.2.2]oct-3-yl]-6-ethynylpyrrolo[1,2-a]pyrazine-3-carboxamide,
or a pharmaceutically acceptable salt thereof.

16. A method for treating a disease or condition in a mammal, wherein the α7 nAChR is activated and the 5-HT₃ receptor is inactivated comprising administering to a mammal a therapeutically effective amount of compound of claim 1.

17. The method according to claim 16, wherein the disease or condition is schizophrenia or psychosis.

18. The method according to claim 17, wherein the mammal would receive symptomatic relief from the administration of a therapeutically effective amount of α7 nAChR agonist/5-HT₃ antagonist and an anti-psychotic agent for a therapeutically effective interval.

19. The method according to claim 16, wherein the disease or condition is cognitive and attention deficit symptoms of Alzheimer's, neurodegeneration associated with diseases such as Alzheimer's disease, pre-senile dementia, senile dementia, traumatic brain injury, behavioral and cognitive problems associated with brain tumors, or Parkinson's disease.

20. The method according to claim 16, wherein the disease of condition is amyotrophic lateral sclerosis, AIDS dementia complex, dementia associated with Down's syndrome, dementia associated with Lewy Bodies, Huntington's disease, attention deficit disorders, attention deficit hyperactivity disorder, depression, anxiety, general anxiety disorder, post traumatic stress disorder, mood and affective disorders including disruptive and oppositional conditions, borderline personality disorder, panic disorder, tardive dyskinesia, restless leg syndrome, Pick's disease, dysregulation of food intake including bulemia and anorexia nervosa, withdrawal symptoms associated with smoking cessation and dependant drug cessation, Gilles de la Tourette's Syndrome, age-related macular degeneration, optic neuropathy, symptoms associated with pain, chemotherapy-induced emesis, migraine, fibromyalgia, irritable bowel syndrome, or diarrhea associated with carcinoid syndrome.

21. The method according to claim 20, wherein the disease or condition is chemotherapy-induced emesis, migraine, fibromyalgia, irritable bowel syndrome, diarrhea associated with carcinoid syndrome, schizophrenia, anxiety, psychosis, restless leg syndrome, pain, glaucoma, age-related macular degeneration, diabetic retinopathy, and withdrawal associated with ceasing the use of drugs, cigarettes, or alcohol upon which one is dependent.

22. The method according to claim 21, wherein the disease or condition is chemotherapy-induced emesis, migraine, fibromyalgia, irritable bowel syndrome, diarrhea associated with carcinoid syndrome, restless leg syndrome, or withdrawal associated with ceasing the use of drugs, cigarettes, or alcohol upon which one is dependent.

23. The method according to claim 22, wherein the disease or condition is chemotherapy-induced emesis, migraine, fibromyalgia, irritable bowel syndrome, or diarrhea associated with carcinoid syndrome.

24. A pharmaceutical composition comprising a compound of claim 1 and a pharmaceutically acceptable excipient, and optionally an anti-psychotic agent.

25. The pharmaceutical composition according to claim 24, wherein said compound and said agent are to be independently administered rectally, topically, orally, sublingually, or parenterally for a therapeutically effective interval.

26. The pharmaceutical composition according to claim 24, wherein said compound is administered in an amount of from about 0.001 to about 100 mg/kg of body weight of said mammal per day.

27. The pharmaceutical composition according to claim 24, wherein said compound is administered in an amount of from about 0.1 to about 50 mg/kg of body weight of said mammal per day, or any range therein.

28. The pharmaceutical composition according to claim 24, comprising a compound of claim 1 and a pharmaceutically acceptable excipient.

29. The pharmaceutical composition according to claim 28, wherein said compound is administered rectally, topically, orally, sublingually, or parenterally for a therapeutically effective interval.

30. The pharmaceutical composition according to claim 28, wherein said compound is administered in an amount of from about 0.001 to about 100 mg/kg of body weight of said mammal per day.

31. The pharmaceutical composition according to claim 28, wherein said compound is administered in an amount of from about 0.1 to about 50 mg/kg of body weight of said mammal per day, or any range therein.
